(19) United States
(12) Patent Application Publication
(10) Pub. No.: US 2023/0260598 A1
(43) Pub. Date: Aug. 17, 2023

(54) APPROACHES TO NORMALIZING GENETIC INFORMATION DERIVED BY DIFFERENT TYPES OF EXTRACTION KITS TO BE USED FOR SCREENING, DIAGNOSING, AND STRATIFYING PATIENTS AND SYSTEMS FOR IMPLEMENTING THE SAME

(71) Applicant: AIONCO, Inc., Menlo Park, CA (US)

(72) Inventors: Shun H. Yip, Cupertino, CA (US); Edmund Wong, Flushing, NY (US); Michael Diamreyan, Rancho Cucamonga, CA (US); Raghuraman Ramamurthy, San Mateo, CA (US); Sidney Tobias, Redwood City, CA (US); Hao Cheng, Santa Clara, CA (US); Victor Solovyev, San Francisco, CA (US)

(21) Appl. No.: 18/168,549

(22) Filed: Feb. 13, 2023

Related U.S. Application Data
(60) Provisional application No. 63/309,991, filed on Feb. 14, 2022.

(51) Int. Cl. G16B 40/20; G06F 16/33

(52) U.S. Cl. CPC G16B 40/20; G06F 16/3334

(57) ABSTRACT

Introduced here is an approach that can be implemented by a computing system to remove kit-specific signals from genetic information to be analyzed, such that cancer presence, progression, or regression can be predicted in an improved manner. The computing system can “preprocess” the genetic information so that diagnoses can be more accurately predicted in a largely, if not entirely, kit-agnostic manner. The computing system may apply one or more models to genetic information as part of an inferencing operation in order to produce one or more outputs, each of which may be indicative of a proposed diagnosis for the corresponding individual. “Preprocessing” could also be performed on the genetic information that is used to train these models, such that the kit-specific signals are removed before a training operation is completed.

### CROSS-REFERENCE TO RELATED APPLICATIONS

This application claims priority to U.S. Provisional Application No. 63/309,991, titled “Normalizing Genetic Information Derived by Different Extraction Kits for Machine Learning-Based Cancer Screening” and filed on Feb. 14, 2022, which is incorporated by reference herein in its entirety.

### TECHNICAL FIELD

Various implementations concern computer programs and associated computer-implemented techniques for normalizing sequenced information, such as text-based representations of genetic information, for training of machine learning models.

### BACKGROUND

Genes are pieces of deoxyribonucleic acid (DNA) inside cells that indicate how to make the proteins that the human body needs to function. At a high level, DNA serves as the genetic “blueprint” that governs operation of each cell. Genes can not only affect inherited traits that are passed from a parent to a child, but can also affect whether a person is likely to develop diseases like cancer. Changes in genes—also called “mutations”—can play an important role in the physiological conditions of the human body, such as in the development of cancer. Accordingly, genetic testing may be leveraged to detect such physiological conditions or likely onsets thereof.

The term “genetic testing” may be used to refer to the process by which the genes or portions of genes of a person are examined to identify mutations. There are many types of genetic tests, and new genetic tests are being developed at a rapid pace. While genetic testing can be employed in various contexts, it may be used to detect mutations that are known to be associated with cancer.

Genetic testing could also be employed as a means for addressing or treating the physiological condition. For example, after a person has been diagnosed with cancer, a healthcare professional may examine a sample of cells to look for changes in the genes to track the progression of the cancer, the efficacy of the treatment, etc. These changes may be indicative of the health of the person (and, more specifically, progression or regression of the cancer). Insights derived through genetic testing may provide information on the prognosis, for example, by indicating whether treatment has been helpful in addressing the mutation.

Implementing computing technologies for the genetic testing may yield valuable insights. For example, artificial intelligence (AI) and machine learning (ML) may be leveraged to analyze DNA information for detecting and/or addressing cancers or potential onset of cancers. However, the magnitude of the DNA information, large number of potential mutations, and large number of samples—among other factors—often negatively impact the effectiveness, accuracy, and practicality in leveraging such computing technologies for the genetic testing.

### BRIEF DESCRIPTION OF THE DRAWINGS

This patent or application publication contains at least one drawing executed in color. Copies of this patent or application publication with color drawings will be provided by the Office upon request and payment of the necessary fee.

FIGS. 1A and 1B show example operating environments of a computing system including a genetic information processing system in accordance with one or more implementations of the present technology.

FIG. 1C shows a flow chart of a method of operating a genetic information processing system that includes a general-sample analysis mechanism in accordance with one or more implementations of the present technology.

FIG. 2 shows an example data processing format for the genetic information processing system in accordance with one or more implementations of the present technology.

FIGS. 3A and 3B show examples of unique segments and refinements thereof in accordance with one or more implementations of the present technology.

FIG. 4 shows example expected phrases in accordance with one or more implementations of the present technology.

FIG. 5 shows example derived phrases in accordance with one or more implementations of the present technology.

FIG. 6 shows an example analysis template in accordance with one or more implementations of the present technology.

FIG. 7 shows an example control flow diagram illustrating the functions of the processing system in accordance with one or more implementations of the present technology.

FIG. 8 shows a flow chart of a method for processing and refining DNA-based text data for cancer analysis in accordance with one or more implementations of the present technology.

FIG. 9 shows a flow chart of a method that, when implemented by the processing system, provides for removal of kit-specific signals from DNA-based text data.

FIG. 10A shows how the processing system can filter “bad” samples using a histogram that plots samples based on the proportion of TR sequences with reads.

FIG. 10B illustrates how low-quality samples outside of the quality range can be filtered from the DNA-based text data being processed by the processing system.

FIG. 11 illustrates how TR sequences can be selected for adjustment by the processing system.

FIG. 12 includes an example of a t-distributed stochastic neighbor embedding plot with samples colored according to extraction kit.

FIG. 13 includes a plot that illustrates read counts for two extraction kits.

FIG. 14 illustrates how all of the non-zero counts across the samples can be converted into values of one, so as to binarize the read counts.

FIG. 15 shows how discovery rate can be computed by the processing system on a per-kit basis across different subsets of samples.

FIG. 16 shows another t-distributed stochastic neighbor embedding plot with samples colored based on extraction kit.

FIG. 17 is a block diagram illustrating an example of a computing system in accordance with one or more implementations of the present technology.

Various features of the technology described herein will become more apparent to those skilled in the art from a study of the Detailed Description in conjunction with the drawings. Various implementations are depicted in the drawings for the purpose of illustration. However, those skilled in the art will recognize that alternative implementations may be employed without departing from the principles of the technology. Accordingly, although specific implementations are shown in the drawings, the technology is amenable to various modifications.

### DETAILED DESCRIPTION

Genetic testing may be beneficial for diagnosing and treating cancer. For example, identifying mutations that are indicative of cancer (1) healthcare professionals make appropriate decisions, (2) researchers direct their investigations, and (3) developers design better therapies, particularly through precision medicine. However, discovering these mutations tends to be difficult, especially as the number of cancers of interest (and thus, corresponding data) increases. Note that the term “mutation,” as used herein, may be used to refer to any change in a DNA sequence. Mutations may not only occur in genes but also intergenic regions and non-coding regions.

To determine whether cancer is present, a processing system may example, in a text-based representation of genetic information (also called “DNA information”), the nucleotides at different molecular positions to identify specific patterns, such as unique segments of repeated characters (e.g., tandem repeats (TRs) corresponding to sequences of two or more DNA bases that are repeated numerous times in a head-to-tail manner on a chromosome, phrases surrounding the unique segments, or derivations thereof that are indicative of mutations. At a high level, the processing system may identify mutations that are indicative of cancer through the discovery of anomalous character sequences at certain molecular positions in the text-based representation of the genetic information. These molecular positions may be representative of target locations in the human genome at which to search for mutations that are indicative of cancer. While the processing system may be able to identify nucleic acid sequences (or simply “sequences”) worth further analysis is an automated matter, there are drawbacks.

One issue is that the results can be influenced by the extraction kit used to extract the genetic information of interest. Extraction kits (also referred to as “capture kits”) include sets of reagents that can be used to extract DNA fragments from a sample taken from a human body. A wide variety of extraction kits with different sets of reagents are available. With different sets of reagents, extraction kits can vary in the principles, procedures, or methodologies that are employed to derive genetic information from a sample. Simply put, different extraction kits are able to more deeply and accurately derive different sequences in the human genome.

This influences the analysis that is performed by the processing system. Assume, for example, that the processing system is tasked with determining whether evidence of a given cancer exists in a given dataset by examining the corresponding genetic information at a set of “N” molecular positions (e.g., Molecular Position 1, Molecular Position 2, Molecular Position 3, . . . , Molecular Position N). Moreover, assume that the given dataset is derived using an extraction kit that struggles to accurately capture genetic information corresponding to a molecular position (e.g., Molecular Position 1) included in the set. In such a scenario, the processing system may erroneously determine that no evidence of the given cancer exists based on the incomplete genetic information.

Introduced here is an approach that can be implemented by a computing system to remove kit-specific signals from genetic information to be analyzed, such that cancer presence, progression, or regression can be predicted in an improved manner. The computing system can “preprocess” the genetic information so that diagnoses can be more accurately predicted in a largely, if not entirely, kit-agnostic manner. As further discussed below, the computing system may apply one or more models to genetic information as part of an inferencing operation in order to produce one or more outputs, each of which may be indicative of a proposed diagnosis for the corresponding individual. “Preprocessing” could also be performed on the genetic information that is used to train these models, such that the kit-specific signals are removed before a training operation is completed.

Implementing the approach may result in improvements across different aspects of mutation discovery, and there are several notable improvements worth mentioning. Without accounting for differences between extraction kits, analyses of genetic information can be incorrect due to misrepresentation or underrepresentation of portions of the human genome in the genetic information. For example, a computing system may predict that an individual (also called a “patient” or “subject”) has the wrong cancer because one or more mutations are not discovered in molecular positions that correspond to misrepresented or underrepresented portions of the human genome. Simply put, if a portion of the human genome cannot be fully represented by a given capture kit, any mutations corresponding to the portion of the human genome will be misrepresented or underrepresented, and such mischaracterization can influence how the computing system understands the health of the corresponding individual. Advantageously, the approach allows discrepancies across different extraction kits to be lessened, thereby reducing the number of incorrect predictions. By adding kit biases, the performance of a model—which may be trained using preprocessed genetic information and/or applied to preprocessed genetic information—can be improved by 10-40 percent. The approach allows somatic mutations in DNA that ultimately lead to the development or progression of different cancers to be more accurately and consistently identified, without concern over which extraction kit was used to sequence the DNA. The amount of improvement may be based on the type of cancer, as the molecular positions of interest can be different for different types of cancer as further discussed below.

Accordingly, kit biases may be added, either as part of a training operation or an inferencing operation, in order to address the misrepresentation or underrepresentation—collectively referred to as “mischaracterizations”—of portions of the human genome. Consider a scenario where, as part of a training operation, a model is trained to identify a given cancer type using genetic information that is associated with a single type of extraction kit, but that type of extraction kit tends to mischaracterize a portion of the human genome in which mutation(s) indicative of the given cancer type occur. Without addressing the mischaracterization, the model may learn to predict occurrences of the given cancer type without meaningful analysis of that portion of the human genome, and thereby leading to inaccurate predictions. Accordingly, mischaracterizations are not only harmful from the data science and ML perspectives, but can also affect how individuals are screened, diagnosed, and further tested—and therefore, whether the appropriate treatment is assigned to address cancer.

Implementations may be described in the context of instructions that are executable by a computing system for the purpose of illustration. However, those skilled in the art will recognize that aspects of the technology described herein could be implemented via hardware or firmware instead of, or in addition to, software. As an example, a computer program that is representative of a software-implemented genetic information processing platform (or simply “processing platform”) designed to process genetic information may be executed by the processor of a computing system. This computer program may interface, directly or indirectly, with hardware, firmware, or other software implemented on the computing system. Moreover, this computer program may interface, directly or indirectly, with computing devices that are communicatively connected to the computing system. One example of a computing device is a network-accessible storage medium that is managed by a healthcare entity (e.g., a hospital system or diagnostic testing facility).

### Overview of Genetic Information Processing System

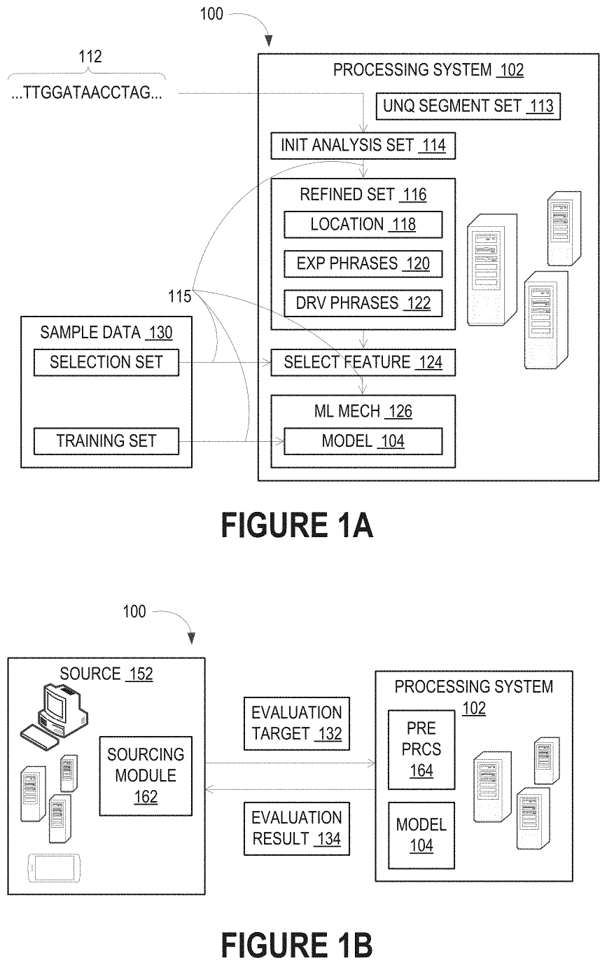

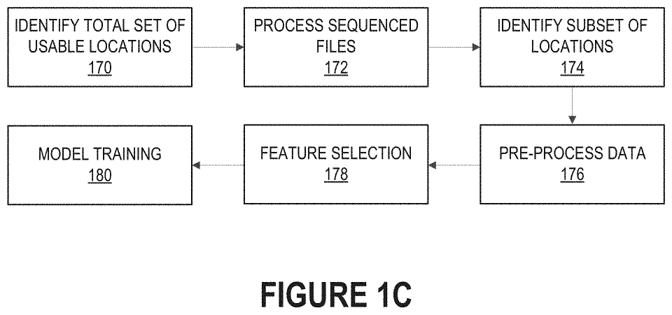

FIGS. 1A and 1B show example operating environments of a computing system 100 including a genetic information processing system 102 (or simply “processing system 102”) in accordance with one or more implementations of the present technology. The processing system 102 can include one or more computing devices, such as servers, personal devices, enterprise computing systems, distributed computing systems, cloud computing systems, and/or the like. The processing system 102 can be configured to analyze DNA information diagnosing one or more types of cancer, for evaluating development stages leading up to the onset of the one or more types of cancer, and/or for predicting a likely onset of the one or more types of cancer.

The operating environment depicted in FIG. 1A can represent a development or training environment in which the processing system 102 develops and trains an analysis mechanism, such as an ML model 104, configured to detect a presence, a progress, or a likely onset of one or more types of cancer. In developing and training the ML model 104, the processing system 102 can first identify an analysis template (e.g., specific data locations or values within reference data 112, such as the human genome or other data derived from human/patient DNA) targeted for further analysis and/or consideration.

As an illustrative example, the processing system 102 can use a text-based representation (e.g., one or more text strings) of the human DNA as the reference data 112. The processing data 102 can analyze the reference data 112 to identify specific locations and/or corresponding text sequences that can be utilized as identifiers or comparison points in subsequent processing. In some implementations, the processing system 102 can use a set of unique text segments 113 (e.g., a set of unique TRs) found or expected in the reference data 112 to generate an initial analysis set 114. The processing system 102 can generate the initial analysis set 114 by identifying expected phrases 120 that include the unique segment set 113 and/or by computing derivations thereof (e.g., derived phrases 122) that represent mutations targeted for analysis. The initial analysis set 114 and/or the unique segment set 113 can include location identifiers 118 associated with a relative location of such segments, phrases, and/or derivations within the reference data 112.

The processing system 102 can further use a refinement mechanism 115 (e.g., a software routine or a set of instructions) that further operates on the initial analysis set 114 and/or subsequent data processing. The refinement mechanism 115 can filter results of one or more data processing operations leading up to the designing and/or training of the ML model 104. The refinement mechanism 115 can generate the filtered result of the initial analysis set 114 as the refined set 116. Additionally or alternatively, the refinement mechanism 115 may be configured to filter during or after the feature selection process and/or the sample data 130.

In some implementations, the refinement mechanism 115 can process the unique segment set 113 and/or the initial analysis set 114 to generate a refined set 116. For example, the refinement mechanism 115 can be configured to remove (1) overlapping TRs from the unique segment set 113, (2) remove duplicated phrases from the initial analysis set 114, (3) filter or adjust for the sample data 130 (e.g., text-based DNA data representative of healthy individuals, cancerous tissues, and/or non-cancerous tissues collected from cancer patients) used to develop and/or train the ML model 104, and/or (4) adjust for, or filter, physiological noise or processing noise. Details regarding the derivation of the initial template and refinement thereof are described below.

For the feature selection, the processing system 102 can iteratively add or remove one or more unique locations/sequences and/or derivations from the refined set 116 and calculate a correlation or an effect of the removed datapoint on the known classifications of the sample data 130 (e.g., to accurately recognize the different categories of the sample data 130). The processing system 102 can determine a set of selected features 124 that correspond to the unique locations/sequences and derivations thereof having at least a threshold amount of effect or correlation with one or more corresponding cancer types. In other words, the processing system 102 can determine the set of features 124 including locations, sequences, mutations, or combinations thereof that are deterministic or characteristic of, or commonly occurring in, corresponding cancers. Based on the set of features 124, the processing system 102 can implement an ML mechanism 124 (e.g., a support vector machine (SVM), a random forest, neural network, etc.) to generate the ML model 104. The processing system 102 can further train the ML model 104 using training data.

Using the refined results, the processing system 102 can limit the amount of data considered or processed in subsequent analyses, such as in feature selection, model generation, model training, and/or the like. For example, the processing system 102 can use the refinement mechanism 115 to reduce the size of the unique segment set 113, thereby reducing the expected phrases 120 and the derived phrases 122 that correspond to the unique segment set 113. Also, the processing system 102 can use the refinement mechanism 115 to further reduce the size of the initial analysis set 114, such as by removing potential duplicated phrases (e.g., across expected/derived phrases at different locations). Accordingly, the processing system 102 can reduce the resource consumption through the reduced size of the refined set 116 (e.g., in comparison to the initial analysis set 114) and reduce the noises and other negative impacts generated by the overlapping/duplicative phrases. Additional sample-, process-, or physiology-based refinement can further increase the overall performance and accuracy of the resulting ML model 104.

The operating environment depicted in FIG. 1B can represent a deployment environment in which the processing system 102 applies the analysis mechanism to detect a presence, a progress, and/or a likely onset of one or more types of cancer from an evaluation target 132 (e.g., a text-based form of patient DNA data). The processing system 102 can generate an evaluation result 134 based on testing the evaluation target 132 with the ML model 104. The processing system 102 can generate the evaluation result 134 that represents a cancer diagnosis or a cancer signal. For example, the evaluation result 134 can represent a determination that the patient has cancer, a stage (e.g., clinically recognized stages 1-4) of the onset cancer, a progress state before, or leading up to, an onset of caner, a likelihood of developing cancer within a predetermined period, an identification of the type of cancer, or a combination thereof.

As an illustrative example, the processing system 102 can include a sourcing device 152 that provides the evaluation target 132 and/or receives the evaluation result 134. The sourcing device 152 can be operated by a patient submitting the evaluation target 132, a healthcare service provider associated with the patient, an insurance company, or the like. Some examples of the sourcing device 152 can include a personal device (e.g., a personal computer or a mobile computing device, such as a wearable device, smart phone, or tablet), a workstation, an enterprise device, etc.

In some implementations, the processing system 102 can include a sourcing module 162 that operates on the sourcing device 152. The sourcing module 162 can include a device, circuit, or a software module (e.g., a codec, application program, or the like) that generates or pre-processes the evaluation target 132. For example, the sourcing module 162 can include a homomorphic encoder that encrypts and prevents unauthorized access to the patient data. The evaluation target 132 can include the homomorphically encoded data that can be processed at the processing system 102 without fully decrypting and recovering the patient data. In other words, the processing system 102 can apply the ML model 104 that is configured to process or perform computations on the encrypted data.

The processing system 102 can include a pre-processing module 164 that conditions the evaluation target 132 for and/or during application of the ML model 104. For example, the pre-processing module 164 can include a device, circuit, or a software module (e.g., a codec, application program, or the like) that removes biases or noises introduced before receiving the evaluation target 132 and/or during the processing (e.g., bootstrapping module to remove noise or other uncertainties introduced by processing encrypted data) of the evaluation target 132.

FIG. 1C shows a flow chart of a method of operating the processing system 102 in accordance with one or more embodiments of the present technology. As discussed above, the ML model 104 can be developed based on identifying a total set of usable locations—represented by the unique segment set 113—within the DNA as shown in block 170. Thus, the total set of usable locations can include a listing of usable TRs and/or molecular locations (also called “genomic locations”) learned through analysis of the reference data 112. After the total set of usable locations are identified, the processing system 102 can process sample data that includes a text-based representation of DNA corresponding to different types of cancers. Said another way, the processing system 102 may process one or more files that include genetic information as shown at block 172. These files may be called “sequenced files,” “DNA files,” or “genomic files” for convenience.

Using the sequenced files, the processing system 102 may produce a filtered version of the total set—represented by the initial analysis set 114 or refined set 116—by identifying the most relevant locations included in the total set of usable locations as shown at block 174. For example, the processing system 102 may process and/or filter the sample data to identify a subset of the molecular locations and corresponding TR sequences and/or read counts (or simply “reads”) included in the total set. In other words, the list of usable locations can be further reduced using various parameters, so as to further increase the processing speed and reduce the number of necessary probes. The resulting subset—namely, the refined set 116—can be processed to remove biases (e.g., capture kit bias), unqualified samples, or the like. For example, the refined set 116 may be “pre-processed” (block 176) prior to feature selection (block 178) and training (block 180) of the ML model 104. Thus, the pre-processing results can be used as part of a training operation, for example, as part of feature selection or training itself, or as part of an inferencing operation. Details regarding how the ML model 104 can be generated, trained, and implemented are provided above and below.

### Data Processing Formats

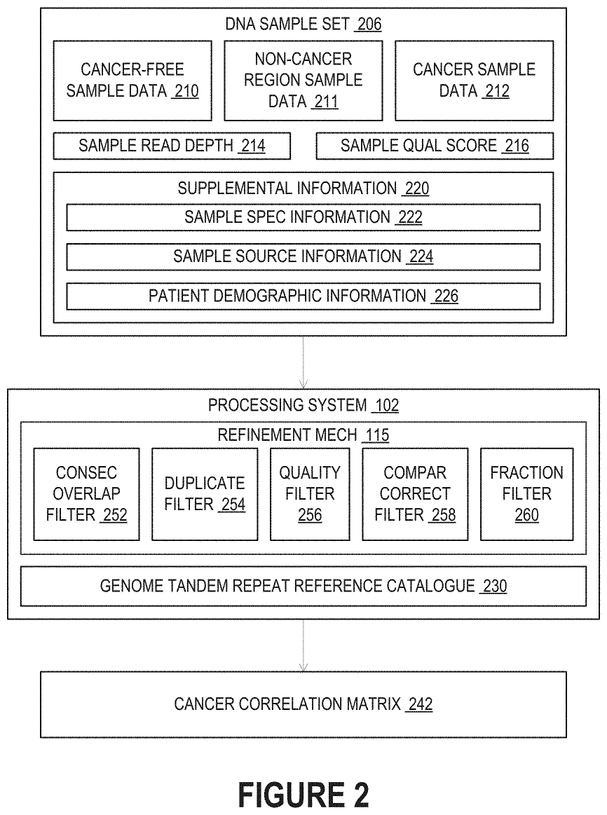

In developing and training the ML model 104 and/or deploying the ML model 104, the processing system 102 can utilize a variety of data processing formats (e.g., data structures, organizations, inputs and outputs, or the like). FIG. 2 shows an example data processing format for the processing system 102 in accordance with one or more implementations of the present technology. The processing system 102 receive and process a DNA sample set 206 (e.g., an instance of the reference data 112 and/or sample data 130 illustrated in FIG. 1A) having one or more of the formats or subfields illustrated in FIG. 2. Moreover, the processing system 102 can generate the initial analysis set 114 (FIG. 1A) and the refined set 116 (FIG. 1A) using one or more detailed example aspects illustrated in FIG. 2.

As an illustrative example, the DNA sample set 206 can include DNA data (e.g., representative of a set of sequenced DNA information) corresponding to different known categories. Examples of the DNA sample set 206 can include genetic information (e.g., text-based representations) derived or extracted from human bodies, such as from tissue extracted during a biopsy or from cell-free DNA (e.g., DNA that is not encapsulated within a cell) in bodily fluids. The DNA sample set 206 can include DNA data collected from volunteers or participating patients having medically confirmed diagnoses and/or from public or private databases.

The DNA sample set 206 can include data collected from different types and/or categories of samples, such as cancer-free samples (cancer-free sample data 210), samples taken from non-cancerous regions (non-cancer region sample data 211), and/or cancerous samples (cancer sample data 212). The cancer-free sample data 210 (or simply “cancer-free data”) can represent text-based DNA data corresponding to samples collected from patients confirmed/diagnosed to be cancer free. The non-cancer region sample data 211 (also called “non-regional data”) can represent text-based DNA data corresponding to samples collected from non-cancerous regions (e.g., white blood cells or leukocytes) of patients confirmed/diagnosed to have one or more types of cancer. The cancer sample data 212 (also called “cancer-specific data”) can represent text-based DNA data corresponding to samples (e.g., tumor biopsies, liquid biopsies, etc.) collected from cancerous regions or tumors confirmed/diagnosed to be a specified type of cancer. The DNA sample set 206 can include information (e.g., the non-regional data 211 and/or the cancer-specific data 212) corresponding to one or more types of cancers (e.g., breast cancer, lung cancer, colon cancer, and/or the like).

The DNA sample set 206 can further include descriptions regarding a strength or a trustworthiness of the data. For example, the DNA sample set 206 can include a sample read depth 214 and/or a sample quality score 216. The sample read depth 214 can represent a number of times that a given nucleotide in the genome (e.g., certain text string/portion) was detected in a sample. The sample read depth 214 may correspond to a sequencing depth associated with processing fragmented sections of the genome within a tissue sample. The sample quality score 216 can represent a quality of identification of the nucleobases generated by DNA sequencing. In some implementations, the sample quality score 216 can include a Phred quality score.

The DNA sample set 206 can also include supplemental information 220 that describes other aspects of the sample or the source of the data. For example, the supplemental information 220 can include information such as sample specification information 222 (or simply “specification information”), sample source information 224 (or simply “source information”), patient demographic information 226, or a combination thereof.

The specification information 222 can include technical information or specifications about the sequenced DNA associated with the DNA sample set 206. For example, the specification information 222 can include information about the locations 118 (FIG. 1A) within the genome to which the DNA fragments correspond, such as intron and exon regions, specific genes, or chromosomes. Also, the specification information 222 can describe, for example, (1) the process, methods, and instrumentation used to extract and sequence the genetic material, (2) the number of sequencing reads for each sample, or a combination thereof.

The source information 224 can include details regarding the source and/or the categorization of the sample. For example, the source information 224 can include information about the cancer type, the stage of cancer development, the organ or tissue from which the sample was extracted, or a combination thereof.

The patient demographic information 226 can include demographic details about the patient from which the sample was taken. For example, the patient demographic information 226 can include the age, the gender, the ethnicity, the geographic location of where the patient resides/visited, the duration of residence/visitation, predispositions for genetic disorders or cancer development, family history, or a combination thereof.

The processing system 102 can analyze the DNA sample set 206 using the mutation analysis mechanism. Accordingly, the processing system 102 can identify mutations or mutation patterns in specific DNA sequences that can be used as markers to determine the existence, the progress, and/or the developing stages of a particular form of cancer. To identify the relevant mutations, the processing system 102 can detect a set of targeted locations or text patterns (e.g., according to the TRs) within the reference genomes.

The processing system 102 can generate and/or utilize a genome tandem repeat reference catalogue 230 that represents a catalogue or a collection of uniquely identifiable TRs in the human genome. As an example, the genome tandem repeat reference catalogue 230 can be based on a reference human genome (e.g., the reference data 112), such as the GRCh38 reference genome. The uniquely identifiable TRs can include DNA sequences having therein a series of multiple instances of directly adjacent identical repeating nucleotide units or base patterns, such as microsatellite DNA sequences. The base patterns can have a predetermined length, such as one for a repetition of one letter or monomer (e.g., ‘AAAA’) or greater (e.g., three for tetramers, such as ‘ACT’). Such uniquely identifiable TRs can serve as reference sequences (e.g., reference locations within the human genome) or markers for evaluating the DNA sample set 206. Since the DNA sample set 206 may correspond to incomplete DNA fragments, the unique TRs found within the fragments may be used to map the DNA information to the human genome.

The processing system 102 can use the genome tandem repeat reference catalogue 230 to compute the initial analysis set 114. For example, the processing system 102 can use the unique TRs identified in the genome tandem repeat reference catalogue 230 to generate derived strings that represent potential mutations. In some implementations, the processing system 102 can identify text characters preceding and/or following each unique TR and derive the mutation strings that represent one or more types of mutations (e.g., insertion-deletion mutations—also called “indel mutations” or “indels”). Details regarding the initial analysis set 114 (e.g., strings with flanking characters and/or mutation strings) are described below.

The processing system 102 can compare the mutations at the targeted locations/sequences across the different types of DNA sample set 206. Based on the comparison, the processing system 102 can compute a correlation between, or a likely contribution of, the mutations at the targeted locations/sequences and the development of cancer. Accordingly, the processing system 102 may generate a cancer correlation matrix 242 that correlates identified tumorous sequences or text-based patterns to specific types of cancer. For example, the cancer correlation matrix 242 can be an index that includes multiple instances of the uniquely identifiable TRs in the genome TR reference catalogue 230 that, when found to be tumorous, indicate the existence of a particular form of cancer or indicate the possibility that a particular form of cancer will develop.

The processing system 102 can perform the feature selection using the cancer correlation matrix 242, such as by retaining the locations/sequences and/or derived mutation patterns having at least a predetermined degree of correlation to one or more corresponding types of cancer. Using the selected features, the processing system 102 can develop and train the ML model 104 configured to detect, predict, and/or evaluate development or onset of cancer.

In some implementations, the processing system 102 can further use the refinement mechanism 115 to generate the refined set 116 (FIG. 1A). The refinement mechanism 115 may include one or more filters to enhance the genome TR reference catalogue 230, the initial analysis set 114, and/or corresponding features, such as by removing or adjusting one or more erroneous or unnecessary sequences. For example, the refinement mechanism 115 can include: (1) a consecutive overlap filter 252 configured to remove consecutive or overlapping sequences (e.g., unique TRs) that effectively point to the same location, (2) a duplicate filter 254 configured to remove duplicate sequences, such as between mutation strings at different locations, (3) a quality filter 256 configured to remove/adjust for input sample data, such as based on quality and/or input depth, (4) a comparison correction filter 258 configured to remove computational noise or errors, (5) a physiology-based filter, such as a fraction filter 260, configured to remove or adjust for physiological features and/or collection-based features that interfere with the data processing, or a combination thereof. Details regarding the refinement mechanism 115 is described below.

### Base Text Patterns—Segments

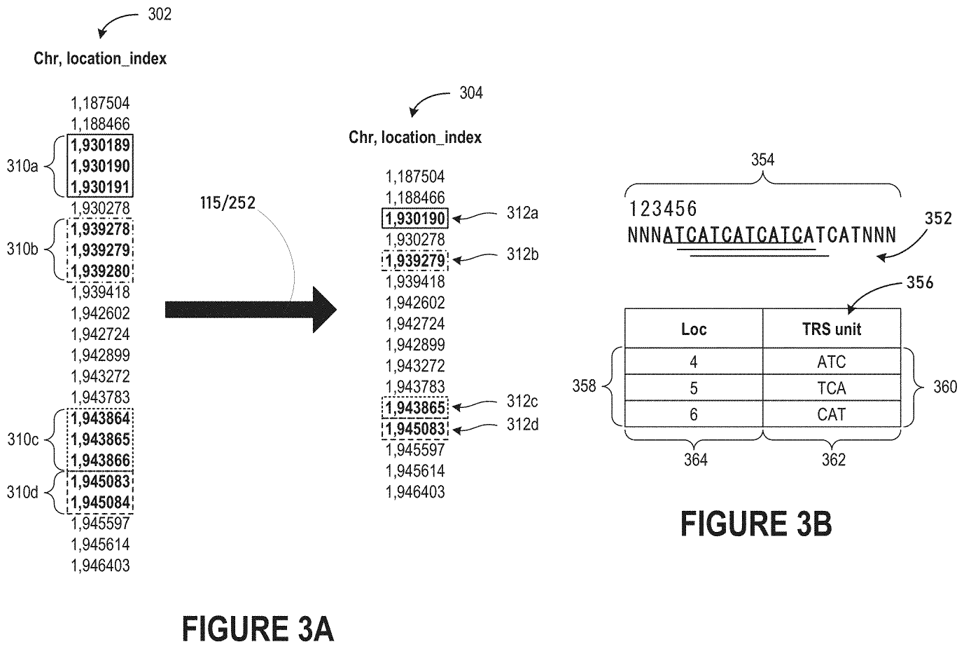

For describing further detailed aspects of the data format, FIGS. 3A and 3B show examples of unique segments (e.g., uniquely identifiable TRs within the human genome) and refinements thereof in accordance with one or more implementations of the present technology. FIG. 3A shows an initial segment set 302 and a refined segment set 304 that correspond to the unique segments 113 of FIG. 1. FIG. 3B illustrates example overlaps 352 in the initial segment set 302. Referring to FIGS. 3A and 3B together, the processing system 102 can use the refinement mechanism 115 (e.g., the consecutive overlap filter 252) to remove the overlaps 352 therein and generate the refined segment set 304.

In some implementations, the processing system 102 can generate the initial segment set 302 based on analyzing the reference data 112 (FIG. 1A) to find uniquely identifiable patterns. For example, the processing system 102 can generate the initial segment set 302 by identifying uniquely identifiable TRs within the human genome. The processing system 102 can use base or TR units (e.g., base character patterns having controllable lengths of one or more characters that are repeated) to identify the overall TR or segment having a corresponding length (e.g., two or more multiples of the TR unit length). The processing system 102 can generate the initial segment set 302 by including repeated patterns of the TRs that exceed a minimum number of base pairs. For example, the repeated TR sequence can be selected based on using the repeated base unit having the minimum number of base pairs ranging between five and eight base pairs.

In the initial segment set 302, the processing system 102 may end up including the overlaps 352 that effectively correspond to a longer and unique string segment and the corresponding location. For the example illustrated in FIG. 3B, a target sequence 354 (e.g., a sequence/combination of nucleotides, such as a portion of the DNA information) can include a uniquely identifiable segment (‘ATCATCATCATCATCAT’ having 17 characters). The processing system 102 can identify unique segments 360 within the target sequence 354 based on identifying repeated adjacent patterns of base units 362. The length of the repeated base units 362 and/or the number of repeats may be predetermined or adjusted in generating the initial segment set 302. For the illustrated example, the targeted segment length corresponds to 12 characters or four repeats of three-letter TR units. Along with the repeated base units 362, the unique segments 360 can be identified based on corresponding segment locations 364 that identify positions (e.g., first letter positions) of the segments within the target sequence 354.

When the target sequence 354 includes a repeated pattern that exceeds the targeted segment length, one target sequence 354 can be identified as including repeats of multiple instances of the base units 356 (e.g., ‘ATC,’ ‘TCA,’ and ‘CAT’). The multiple instances of the base units 356 may correspond to shifted results of each other. As such, the multiple unique segments 360 can overlap each other and/or be sequentially shifted by one or more characters relative to each other. FIG. 3A illustrates a portion of the initial segment set 302 having overlapping location sets 310a, 310b, 310c, and 310d that correspond to such overlapping instances of the unique segments 360. However, given the nature of the overlaps, each of the overlapping location sets 310a, 310b, 310c, and 310d can effectively correspond to a single segment/location rather than the multiple separate segments/locations.

The processing system 102 can use the refinement mechanism 115 to identify and remove the overlaps 352 in the unique segments 360. In some implementations, the consecutive overlap filter 252 can be configured to ensure that the initial segment set 302 is sorted according to the segment location 358. With the sorted segments, the consecutive overlap filter 252 can identify patterns in the segment location 358 of adjacent segments within the initial segment set 302. The consecutive overlap filter 252 can be configured to identify the overlaps 352 when the segment location 358 of the adjacent segments are separated by a predetermined number (e.g., one, two, or more, a number based on the repeated unit length and/or the targeted segment length, and/or the like). Also, the consecutive overlap filter 252 can be configured to identify the overlaps 352 when the segment location 358 follows one or more pattern (e.g., consistently separated by one or two values) over two, three, or more adjacently occurring segments. The consecutive overlap filter 252 can group the two or more adjacent segments that satisfy the separation threshold/pattern as a set of the overlaps.

Additionally or alternatively, the consecutive overlap filter 252 can configured to identify the overlaps 352 when the repeated base units 356 for the adjacent segments correspond to circularly shifted values. For the example illustrated in FIG. 3B, the processing system 102 can identify that the unique segments 360 at locations 4, 5, and 6 correspond to an overlapping set since the repeated base units 356 of ‘ATC,’ ‘TCA,’ and ‘CAT’ correspond to circularly shifting a preceding unit by one character/position. The consecutive overlap filter 252 can group the two or more adjacent segments that satisfy/maintain the detected pattern in the repeated base units 356 a set of the overlaps.

After the sets of overlaps are identified, the consecutive overlap filter 252 can refine the set by reducing the number of overlapped segments. For example, the consecutive overlap filter 252 can retain one segment from each set of overlaps and remove others. In some implementations, the consecutive overlap filter 252 can be configured to select the segment according to a predetermined location, the target segment length, the repeated unit length, or a combination thereof. For example, the consecutive overlap filter 252 can be configured to select the segment positioned in the middle/center of the set. Also, the consecutive overlap filter 252 can include a predetermined equation that identifies the selection location according to the number of segments in the set, the target segment length, the repeated unit length, or a combination thereof. The selected locations can be represented as refined locations (e.g., refined locations 312a, 312b, 312c, and 312d respectively corresponding to overlapping sets 310a, 310b, 310c, and 312d) in the refined segment set 304.

### Base Text Patterns—Expected Phrases

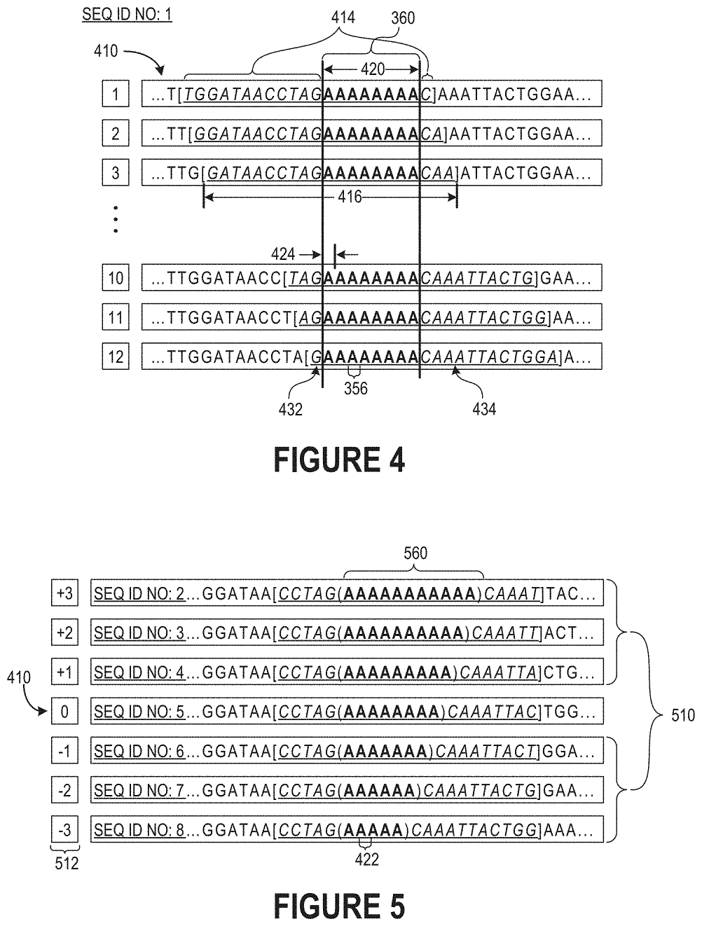

The processing system 102 can use the processed segments (e.g., the refined segment set 304) to generate phrases. FIG. 4 shows example expected phrases 410 in accordance with one or more implementations of the present technology. The expected phrases 410 can correspond to textual representations of the DNA sequences or a set of sequence variations that may be used as bases for subsequent processing/comparing, such as in deriving mutations strings and analyzing the DNA sample set 206 (FIG. 2).

For context, samples collected from patients may include fragments or portions of the overall DNA. As such, the corresponding sequenced values or the text string may include different combinations of characters. The processing system 102 (FIG. 1A) can generate the expected phrases 410 as representations of different character combinations that include the uniquely identifiable segments (e.g., the refined segment set 304 (FIG. 3A), such as the refined set of unique TRs).

Accordingly, the processing system 102 can generate the expected phrases 410 based on the refined segment set 304 instead of the initial segment set 302 (FIG. 3A). In some implementations, the processing system 102 can generate a set (illustrated as a unique sequence identifier number in FIG. 4) of the expected phrases 410 for each of the unique segments 360 (illustrated using bolded characters in FIG. 4) in the refined segment set 304.

The expected phrases 410 can have a phrase length 416 of k (e.g., generally between 10 to 50, but could be greater than 50 or fewer than 10) number of DNA base pairs or pairs of nucleobases. Each DNA base pair can be represented as a single text character (e.g., ‘A’ for adenine, ‘C’ for cytosine, ‘G’ for guanine, and ‘T’ for thymine). As such, the expected phrases 410 may also be referred to as “k-mers.”

In some implementations, as described above, the unique segments 360 can include a DNA sequence of a specified minimum length. A unique segment 360 can include a series of multiple instances of directly adjacent identical repeating nucleotide units or the repeated base units 356. For example, the unique segment 360 can include a minisatellite DNA or microsatellite DNA sequence of a specified minimum length. Accordingly, the unique segment 360 can correspond to a repeated pattern of the repeated base units 356, and the number of repetitions can correspond to a segment length 420 (e.g., the total length of, or total number of, nucleotide base pairs) for the unique segment 360. The repeated base unit 356 can have a base unit length 424 corresponding to the number of nucleotides within the repeated base unit 356 (e.g., one for a mono-nucleotide, two for a di-nucleotide, etc.).

For illustrative purposes, FIG. 4 shows a specific instance for the unique segment 360 of “AAAAAAAA,” annotated as “A8,” located at the molecular position starting at “10,513,372” on chromosome 22. In this example, the unique segment 360 includes the segment length 420 of eight base pairs with the repeated base unit 356 of one base pair (e.g., a monomer or a mono-nucleotide) ‘A.’

The processing system 102 can use the phrase length 416 (e.g., k between 10 to 50 base pairs) that has been predetermined or selected to capture targeted amount of data/characters surrounding the unique segments 360. As such, the phrase length 416 can be greater than the segment length 420, and each of the expected phrases 410 can include a set of flanking texts 414 (e.g., text-based patterns; illustrated using italics in FIG. 4) preceding and/or following the corresponding unique segment 360.

The processing system 102 can generate the expected phrases 410 in a variety of ways. As an illustrative example, the processing system 102 can use each of the unique segments 360 as an anchor for a sliding window having a length matching the phrase length 416. The processing system 102 can iteratively move the sliding window relative to the unique segment 360 and log the text captured within the window as an instance of the expected phrases 410. As such, each of the expected phrases 410 can correspond to a unique position of the sliding window relative to the unique segment 360. Also, the set of expected phrases 410 for one reference TR can include different combinations of the flanking text 414 (e.g., a combination of one or more leading characters 432 and/or one or more tailing characters 434).

The total number of base pairs in flanking text 414 can be a fixed value that is based on the phrase length 416 and the segment length 420. The number of characters in the flanking text 414 can be calculated as the difference between the phrase length 416 and the segment length 420. As an example, for one of phrases having a length of 21 base pairs and a segment length of 8 base pairs, the flanking text can include 13 base pairs.

Each of the expected phrases 410 can represent one of a number of position variant k-mers based on the flanking texts 414. The position variant k-mers can include specific numbers of base pairs in the leading flanking text 432 and tailing flanking text 434. For example, a set of the expected phrases 410 can include the same unique segment (e.g., repeated pattern of the TR) and differ from one another according to the number of base pairs included in the leading flanking text 432 and/or the tailing flanking text 434. In general, the number of base pairs included in the leading flanking text 432 and tailing flanking text 434 can vary inversely between the different instances of the position variant k-mers or expected phrases 410.

As an example, each of the expected phrases 410 illustrated in FIG. 4 has the phrase length 416 of 21 base pairs and the segment length 420 of 8 base pairs. A first expected phrase can have the leading characters 432 corresponding to 12 base pairs and the tailing character 434 corresponding to 1 base pair. A second expected phrase can have the leading characters 432 corresponding to 11 base pairs and the tailing characters 434 of 2 base pairs. The pattern can be repeated until the last expected phrase has the leading characters 432 corresponding to 1 base pair and the tailing characters 434 corresponding to 12 base pairs.

The expected phrases 410 can be grouped into sets that each correspond to a unique segment as described above. The total number of phrases or position variant k-mers (position variant total) in the grouped set can be represented as:

Position Variant Total=(Phrase length k)−(Segment length)−1.

For the example illustrated in FIG. 4, the set of expected phrases can have a position variant total of 12, representing 12 different instances of phrases corresponding to the phrase length 416 of 21 and the segment length 420 of 8.

In some implementations, the processing system 102 can use the unique instances of the TRs as the basis for generating the sets of expected phrases 410. Accordingly, each of the expected phrases 410 can also be unique since it is generated using the corresponding unique TR as a basis. The processing system 102 can use the unique expected phrases 410 to account for and identify the fragmentations likely to be included in the patient samples.

### Base Text Patterns—Derived Phrases

The processing system 102 can use the expected phrases 410 to analyzes mutations in genetic information (e.g., sequenced DNA segments), such as for detecting tumorous/cancerous DNA sequences. The expected phrases 410 can be used to detect locations within the reference genome and related mutations that are indicative of certain types of cancers or likely onset thereof. The processing system 102 can use the expected phrases 410 as basis to generate derived phrases that represent various mutations in the genetic information. The processing system 102 can use the derived phrases to recognize or detect mutations in the DNA sample set 206 (FIG. 2), the sample data 130 (FIG. 1A), or the like in developing, training, and/or deploying the ML model 104. Effectively, the processing system 102 can identify the mutation patterns indicative of certain types of cancers based on using the derived phrases to determine differences between healthy and cancerous DNA samples (e.g., between the cancer-free data 210, the non-regional data 211, and/or the cancer-specific data 212 illustrated in FIG. 2).

FIG. 5 shows example derived phrases 510 in accordance with one or more implementations of the present technology. The processing system 102 (FIG. 1A) can generate the derived phrases 510 based on adjusting the expected phrases 410 expected to a predetermined pattern. For example, for one or more or each of the expected phrases 410, the processing system 102 can generate a set of the derived phrases 510 that represent indel mutations of the corresponding expected phrase 410. In some implementations, the processing system 102 can generate the set of derived phrases 510 that correspond to a predetermined number of insertions and/or deletions in the unique segment 360 (FIG. 4) within the corresponding expected phrase 410. In other words, the set of derived phrases 510 can represent the indel variants of the sequence represented by the corresponding expected phrase 410.

The processing system 102 can generate the set of the derived phrases 510 based on adjusting (via insertion/deletion) the number of the repeated base units 356 (FIG. 4) and/or one or more characters in the unique segment 360 of the expected phrase 410. Accordingly, the processing system 102 can generate a set of derived segments 560 that correspond to indel variants of the unique segment 360.

The processing system 102 can generate the derived phrases 510 based on adding and/or adjusting the flanking text 414 (FIG. 4) around the derived segments 560 (illustrated as the bolded characters within parentheses ‘( )’). In some implementations, the processing system 102 can generate the derived phrases 510 having the same phrase length 416 (FIG. 4) as the expected phrases 410. As a result, the processing system 102 can expand or reduce the coverage of the flanking text 414 according to the indel changes to the unique segment 360 (e.g., the originating pattern of TRs). With deletions, the processing system 102 can include corresponding number of new characters from the overall sequence into the flanking text 414 (FIG. 4). Similarly with additions, the processing system 102 can remove the corresponding number of characters from the flanking text 414. For illustrative purposes, FIG. 5 shows the surrounding adjustments occurring in the trailing characters 434 (FIG. 4) while maintaining the leading characters 432 (FIG. 4). However, it is understood that the processing system 102 can operate differently, such as by (1) adjusting the leading characters 432 while maintaining the trailing characters 434 and/or (2) spreading the adjustments across the leading characters 432 and the trailing characters 434 according to the number of characters in the original phrase and/or a predetermined pattern.

For the example illustrated in FIG. 5, the expected phrase 410 can correspond to the repeated TR sequence of “AAAAAAAA” or A8 beginning at position 10,513,372 on chromosome 22. The derived phrases 510 can correspond to the derived segments 560 including up to three insertions and deletions of the repeated base unit ‘A.’ In other words, the derived phrases 510 can correspond to phrases built around A5, A6, A7, A9, A10, and A11.

The number of the derived phrases 510 associated with a given expected phrase can be determined by an indel variant value 512. The indel variant value 512 can include an integer value representative of the number of insertions and deletions. The indel variant value 512 can further function as an identifier for a phrase. For example, the indel variant value ‘0’ can represent the expected phrase 410 having zero insertions/deletions. Positive indel variant values (e.g., 1, 2, 3) can represent derived phrases including corresponding number of insertions of base units or characters in the repeated TR portion. Negative indel variant values (e.g., −1, −2, −3) can represent derived phrases corresponding number of deletions of base units or characters in the repeated TR portion. For the example illustrated in FIG. 5, the indel variant values 1, 2, and 3 can represent/identify A9, A10, and A11, respectively. Also, the indel variant values −1, −2, and −3 can represent A7, A6, and A5, respectively.

For context, the processing system 102 can use the expected phrases 410 and the corresponding sets of derived phrases 510 to analyze the DNA sample set 206 and develop/test the ML model 104 (FIG. 1A). The phrases generated using the unique TR patterns can provide accurate and precise identification of corresponding sequences in the different types of health and cancerous DNA samples. In other words, the various phrases can represent the type of textual patterns or the corresponding sequences that are targeted for analyses and comparisons between the cancer-free data 210, the non-regional data 211, and/or the cancer-specific data 212. For example, the processing system 102 can use the various phrases to identify the numbers and types/locations of mutations in the cancer-related samples and absent in healthy samples. The processing system 102 can aggregate the results across multiple samples and patients to derive a pattern or a correlation between certain types of mutations and the onset of certain types of cancer.

To put things another way, the processing system 102 can identify unique patterns (e.g., the unique TR patterns and/or the corresponding expected phrases 410) that each occur once within the human genome. The unique patterns can be used to identify specific locations and portions within the human genome for various analyses. Moreover, the processing system 102 can target specific types of mutations, such as indel mutations, in developing a cancer-screening tool and/or a cancer-predicting tool. It has been found that various types of cancers can be accurately detected and progress/status of such types of cancers can be described using the expected phrases 410 and the corresponding sets of the derived phrases 510 (e.g., sequences identified using unique TR-based patterns and indel variants thereof) and without considering other aspects/mutations of the human DNA. As a result, the processing system 102 can generate the ML model 104 that can accurately detect the existence, predict a likely onset, and/or describe a progress of certain types of cancers using the various phrases. In other words, the processing system 102 can detect/predict the onset of cancer without processing the entire DNA sequence and different types of mutation patterns.

The processing system 102 can further improve the efficiency and reduce the resource consumption using the indel variant value 512. Given the downstream processing methodology, the indel variant value 512 can control the number of phrases considered in developing/training the ML model 104 and thereby affect the overall number of computations and the amount of resource consumption. When the indel variant value 512 is too high, the processing system 102 may end up analyzing a reduced or ineffective number of possible sequences. For example, as the total number of base pairs in the TR indel variant approaches the phrase length 416, the number of available derived phrases and the likely occurrence of such mutations decrease. Accordingly, in some implementations, the indel variant value 512 in the range of three to five provides sufficient coverage for varying degrees of possible insertion and deletion mutations that are indicative of one or more types of cancer. This range of values may be sufficient to provide accurate results without requiring ineffective or inefficient amount of computing resources.

Additionally, the processing system 102 can further improve the efficiency and reduce the resource consumption using the segment length 420 (e.g., the length of the uniquely identifiable TR-based pattern). It has been found that the probability of mutation occurrences decreases as the tandem repeat segment length 420 is reduced. In particular, the mutation rate for genome TR sequences with segment length 420 of fewer than five base pairs is significantly less than genome TR sequences with segment length 420 of five or more base pairs. Thus, the expected phrases 410 can be selected as the genome TR sequence with segment length 420 of five or greater.

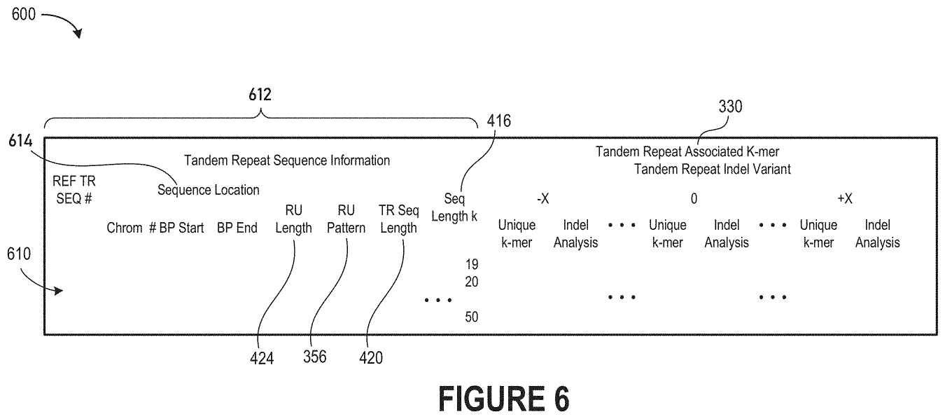

The processing system 102 can store the various phrases (e.g., the expected phrases 410 and/or the corresponding sets of the derived phrases 510) in the genome TR reference catalogue 230 (FIG. 2). FIG. 6 shows an example analysis template 600 in accordance with one or more implementations of the present technology. The processing system 102 can use the analysis template 600 to represent the various phrases and/or track the associated processing results.

In some implementations, the analysis template 600 can correspond to a format for the genome TR reference catalogue 230. The genome TR reference catalogue 230 can include catalogue entries 610 for each instance of the unique segments 360 (e.g., uniquely identifiable TR patterns or reference TR patterns). The entries 610 can include TR sequence information 612 that characterizes the unique segments 360 and/or the derived segments 560. For example, the TR sequence information 612 can include a sequence location 614, the segment length 420, the base unit length 424, the repeated base unit 356, or a combination thereof.

The sequence location 614 can identify the location of the corresponding unique segment 360 and/or expected phrase 410 within the reference genome. As an example, the sequence location 614 can be described based on the molecular location of the unique segment 360, such as (1) the chromosome on which the TR sequence is located and/or (2) the base pair numbers in the chromosome marking the beginning/ending of the TR sequence. The sequence location 614 can act as a unique identifier that distinguishes one instance of the unique segment 360 and/or the expected phrase 410 from another. For example, expected phrases 410 that share the same repeated base unit 356 and the base unit length 424 can be distinguished from one another based on the sequence location 614.

The entries 610 for each instance of the unique segment 360 can include information for one or more instances of the corresponding phrases (e.g., expected and/or derived). For example, the entries 610 can include information for the expected phrases 410 and/or the derived phrases 510 with various values for the phrase length 416. For illustrative purposes, this instance of entries 610 is shown including information for the expected phrases 410 with phrase lengths corresponding from 19 base pairs to 60 base pairs. However, it is understood that the entries 610 can include information regarding expected phrases 410 with fewer than 19 base pairs and/or greater than 60 base pairs. As another example, the entries 610 can include information that distinguishes between the expected phrases 410 and the derived phrases 510. In some implementations, the entries 610 can identify expected phrases 410 associated with a corresponding TR pattern. For instance, the TR pattern of ‘A8’ beginning at position 10,513,372 can yield 16 sequences or expected phrases 410 having the phrase length 416 of 30 base pairs.

The entries 610 can further identify the derived phrases 510 that are absent from the reference genome. For illustrative purposes, Table 1 below summarizes the derived phrases 510 having the segment length 416 of 30 base pairs for the unique segment 360 or TR pattern of ‘A8’ beginning at position 10,513,372 (annotated as '372) on chromosome 22. In this example, each of the derived phrases 510 corresponding to indel variants with the indel variant value 512 ranging from “−5” to “+5” are not found in the reference genome.

TABLE 1
Chromosome 22, ‘372, “A8” Reference
TR Associated Indel Phrase Summary

| | | Total That Do |
|---|---|---|
| Indel Variant Value | Position Variant Total | Not Appear |
| +5 | 16 | 16 |
| +4 | 17 | 17 |
| +3 | 18 | 18 |
| +2 | 19 | 19 |
| +1 | 20 | 20 |
| −1 | 22 | 22 |
| −2 | 23 | 23 |
| −3 | 24 | 24 |
| −4 | 25 | 25 |
| −5 | 26 | 26 |

The analysis template 600 can be used to track the statistical data generated during development/training of the ML model 104. For example, the processing system 102 can track the occurrences of certain mutations according to the sequence location 614 or the identifier for the corresponding entry 610 and the indel mutation offset/identifier. The processing system 102 can use the counted occurrences for each sample, each sample set, or a combination thereof to compute the correlation between the mutations and the onset of the corresponding type of cancer.

In some implementations, the processing system 102 can calculate the number of occurrences for each of the expected and/or derived phrases, such as for indel variants with or without indel variant ‘0,’ in the patient sequencing data. For each set of phrases associated with a particular indel variant type, the processing system 102 can calculate a statistical value (e.g., a median value) from the set of the number of occurrences. The median value can represent the counts associated with the particular TRS with a particular type of indel variant in the corresponding patient.

As an illustrative example, the processing system 102 can process three TR sequences derived from a targeted k=16 wild-type nucleotide (e.g., ATCATCATC) as shown below in Table 2.

TABLE 2

| TR Sequence Associate | K-mer |
|---|---|
| K-mers (Underlined) | Count |
| . . . ACTTGAATCATCATCATCCTCCTA . . . | 7 |
| . . . ACTTGAATCATCATCATCCTCCTA . . . | 11 |
| . . . ACTTGAATCATCATCATCCTCCTA . . . | 10 |

The processing system 102 can calculate the median value of the counts as 10. Accordingly, the processing system 102 can assign a count of 10 to a corresponding TR sequence indel type (e.g., indel type +1) for this patient.

The analysis template 600 is shown for exemplary purposes as a template with a general layout for organizing information for each of the segments and/or phrases. It is understood that the analysis template 600 can include different categorizations and arrangements with additional or different pieces of information. Further, it is understood that an active or “in use” version of the genome TR reference catalogue 230 can be populated with values corresponding to the various categories of the entries 610.

In addition to carefully selecting the processing parameters (e.g., the indel variant value 512 and/or the segment length 420) and reducing the overlaps 352 in the unique segments 360 described above, the processing system 102 can further increase the processing efficiencies and accuracy of the ML model 104 by removing duplicate phrases or k-mers. The processing system 102 can inadvertently introduce or generate the duplicate phrases since the derived phrases 510 are generated by altering the unique segments 360. In other words, the derived phrases 510 may include character sequences that match other phrases corresponding to other portions of the human genome (e.g., derived and/or unique phrases corresponding to different locations or TR combinations). The processing system 102 can use the refinement mechanism 115 (e.g., the duplicate filter 254 (FIG. 2)) to identify and remove such duplicated phrases.

In some implementations, the duplicate filter 254 can be configured to compare the derived phrases 510 to the expected phrases 410 corresponding to different locations in the human genome. Additionally or alternatively, the duplicate filter 254 can be configured to compare the derived segments 560 to the unique segments 360 associated with other locations. Moreover, the duplicate filter 254 can compare the derived phrases 510 and/or derived segments 560 across different locations to find matches. For example, the processing system 102 can sort the phrases according to the unique segments 360 and/or the repeated base unit 356 and then according to the base unit length 424. The duplicate filter 254 can be configured to remove one or more or all of the instances of the matching phrases (having, e.g., same base TR units and TR-pattern length). In other words, the duplicate filter 254 can remove from further processing character combinations representative of sequences/mutations that can be found at multiple locations in the human genome. Accordingly, the processing system 102 can ignore the potentially misleading character patterns in analyzing for correlations to different types of cancers and reduce the overall number of processed phrases.

### Downstream Filtering

In addition to the text-based filtering described above, the processing system 102 can further filter the data and/or the processing results. For example, the processing system 102 can use the quality filter 256 (FIG. 2) to preprocess and/or adjust for the input patient data, such as the DNA sample set 206. The processing system 102 can use the quality filter 256 to reduce, remove, or adjust for imperfections (e.g., biases caused by inaccurate/insufficient reads) that may be introduced by sequencing technologies. In some implementations, the quality filter 256 can adjust for or normalize different read depths (e.g., the number of times that a given nucleotide in the genome was detected in a sample) across the separately sequence data, such as across the cancer-free data 210, the non-regional data 211, and/or the cancer-specific data 212.

To adjust for the different read depths, the quality filter 256 can be configured to require minimum read depths for the input patient data. In other words, the quality filter 256 can remove or filter out samples and/or corresponding sequenced strings having the sample read depth 214 (FIG. 2) less than a predetermined threshold (e.g., 10). Additionally or alternatively, the quality filter 256 can be configured to normalize the read depths to a predetermined depth (e.g., 200) across the different data sets. In normalizing the read depth, the quality filter 256 can calculate a scale factor for each data set by dividing the predetermined depth by the corresponding sample read depth 214. The scale factor can be applied or multiplied to wild-type counts (e.g., number of character sequences/segments corresponding to genes found in natural non-mutated form) for the set, thereby calculating the normalized wild-type count. Similarly, the quality filter 256 can apply the scale factor to the mutation counts (e.g., indel counts) found in each corresponding set. Accordingly, the wild-type counts and the mutations counts for the different data sets can be normalized to a common predetermined read depth using the scale factor.

Additionally or alternatively, the quality filter 256 can be configured to remove nucleotides having sub-standard quality. For example, the quality filter 256 can be configured to filter out data samples or strings having the sample quality score 216 (FIG. 2), such as the Phred quality score, below a predetermined quality threshold (e.g., 20). The quality filter 256 can replace characters for the substandard nucleotides to a predetermined character (e.g., ‘N’).

The processing system 102 can further use the comparison correction filter 258 (FIG. 2) to remove computational noise or errors. Even with the reduced number of computations, the number of computations and comparisons may inadvertently introduce false positives. Accordingly, the comparison correction filter 258 can be configured to correct the intermediate data, such as using a Bonferroni correction process. For example, the comparison correction filter 258 can adjust (by, e.g., dividing) a predetermined somatic classification threshold (p-value criteria, such as 0.01) by the number of phrases being processed/compared.

Moreover, the processing system 102 can use the fraction filter 260 (FIG. 2) to remove or adjust for physiological features and/or collection-based features that interfere with the data processing. In some implementations, the fraction filter 260 can be configured to address samples having relatively low numbers of derived phrases (e.g., sample sets having mutant counts less than a predetermined threshold). For example, the fraction filter 260 can include an allelic fraction filter. The allelic fraction for sample/data can be calculated based on dividing the number of derived phrases 510 by a sum of wild-type counts and mutant counts. The fraction filter 260 can classify data/strings as not being somatic when the corresponding allelic fraction values are less than a predetermined threshold (e.g., 0.05).

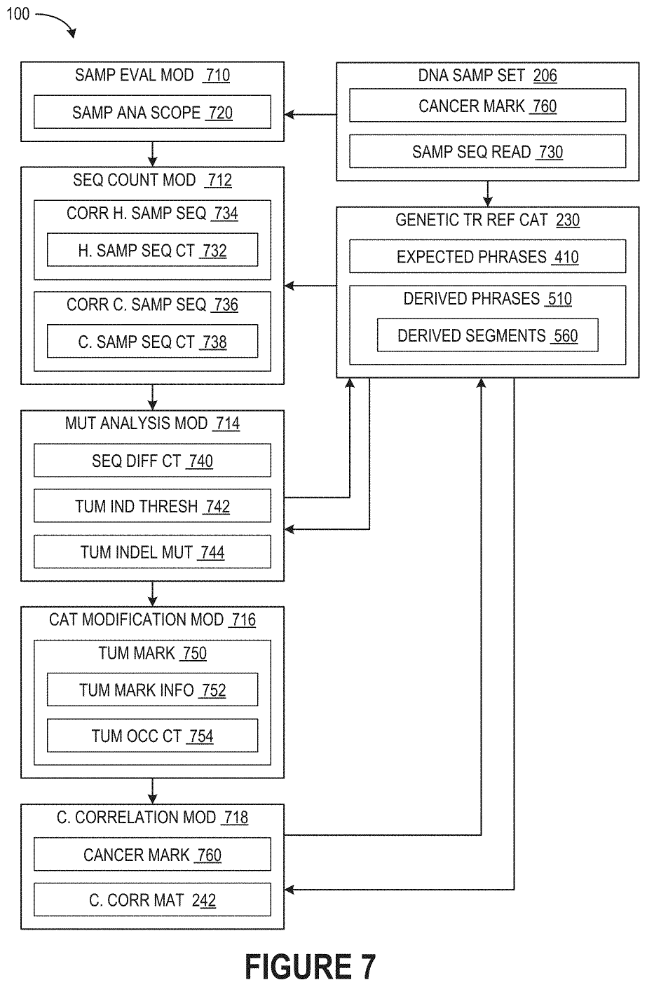

FIG. 7 shows a control flow diagram illustrating the functions of the computing system 100 in accordance with various implementations. The computing system 100 can be implemented to supplement and refine information in the genome TR reference catalogue 230 with information from the DNA sample sets 206 based on the unique segments 360 and the various phrases. In general, the computing system 100 can analyze one or more of the DNA sample sets 206 to process (1) mutations at specific locations of DNA sequences, (2) correlation of mutation patterns, (3) corresponding indications of one or more types of cancer, or a combination thereof. The functions of the computing system 100 can be implemented with a sample set evaluation module 710, a sequence count module 712, a mutation analysis module 714, a catalogue modification module 716, a cancer correlation module 718, or a combination thereof.

The evaluation module 710 can be configured to evaluate the scope of the DNA sample set 206, including the cancer-free data 210, the non-regional data 211, and/or the cancer-specific data 212. For example, the evaluation module 710 can evaluate the DNA sample set 206 to identify factors, properties, or characteristics thereof to facilitate analysis of the different categories of data. In some implementations, the evaluation module 710 can be optional. The evaluation module 710 can generate a sample analysis scope 720 for the DNA sample set 206. The sample analysis scope 720 is a set of one or more factors that may govern/control the analysis of the DNA sample set 206. For example, the sample analysis scope 720 can be generated based on the supplemental information 220. The sample analysis scope 720 can be used to identify usable phrases (e.g., the expected phrases 410 and/or the derived phrases 510) based on the sequence location 614 and the phrase length k 416.

The computing system 100 can receive the derived phrases 510 and associated information from the genome TR reference catalogue 230 and/or the DNA sample set 206. The mutation analysis mechanism can be implemented with the count module 712 and the analysis module 714. The count module 712 may be responsible for calculating a number of occurrences (e.g., a sequence count) for specific DNA sequences/phrases in a sample set. The count module 712 can calculate the sequence count based on a number of sample sequence reads 730, such as the sequence reads for the DNA fragments in one or more categories of data in the DNA sample set 206.

For the cancer-free data 210, the count module 712 can calculate a healthy sample sequence count 732 for each instance of a corresponding healthy sample sequence 734 identified in the cancer-free data 210. The corresponding healthy sample sequence 734 is a DNA sequence in the healthy sample DNA information 734 that corresponds to one of the derived segments 560 and/or the derived phrases 510. The heathy sample sequence count 732 is the number of times that the corresponding healthy sample sequence 734 is identified in the cancer-free data 210. Similarly, for the cancer-specific data 212 and/or the non-regional data 211, the count module 712 can calculate count values for each instance of a targeted sequence identified in the data group. In other words, the count module 712 can calculate the number of times the various phrases are found within the samples according to the corresponding categories.

The count module 712 can identify the corresponding healthy sample sequence 734 and the corresponding cancerous sample sequence 738 for a given expected phrase, and more specifically the derived phrase. For example, the sequence count module 712 can search through the different categories of data for matches to one or more of the derived segments within the corresponding phrases. As one specific example, the count module 712 can search for a string of consecutive base pairs that matches one of the derived segments 560 of the derived phrases 510.

The count module 712 can calculate the healthy sample sequence count 732 as the total number of each of the corresponding healthy sample sequence 734 identified in each of the sample sequence reads 730 in the cancer-free data 210. In many cases, the corresponding healthy sample sequence 734 will correspond with a single instance of the tandem repeat indel variants 310. In these cases, the total value of the healthy sample sequence count 732 will be equal to the total number of the sample sequence reads 730 in the cancer-free data 210. For example, where the cancer-free data 210 includes 50 instances of the sample sequence reads 730 per DNA segment, the healthy sample sequence count 732 for a given instance of the corresponding healthy sample sequence 734 should also be 50. The case of non-unity between the number of sequencing reads and the healthy sample sequence count 732 can generally be attributed to sequencing errors.

In many cases, the corresponding healthy sample sequence 734 will match with the phrase with the indel variant value 312 of zero (e.g., the expected phrase with no insertions or deletions of the unique segment 360). However, in some cases, the corresponding healthy sample sequence 734 can differ. The differences between the corresponding healthy sample sequence 734 and the phrase with the indel variant value 312 of zero can account for wild type variants (e.g., naturally occurring variations) in the cancer-free data 210.

Similarly, the count module 712 can calculate the cancerous sample sequence count 736 for each of the corresponding cancerous sample sequence 738 that appear in the sample sequence reads 730 in the cancer-specific data 212. Due to possible mutations, the cancer-specific data 212 can include multiple different instances of the corresponding cancerous sample sequence 738 matching different instances of the derived segments 560, with each corresponding cancerous sample sequence 738 having varying values of the cancerous sample sequence count 736. As an example, in some cases, the corresponding cancerous sample sequence 738 and cancerous sample sequence count 736 will match with the corresponding healthy sample sequence 734 and healthy sample sequence count 732, indicating no mutations. As another example, for a given instance of the derived phrase 510, the cancer-specific data 212 may have a split in the cancerous sample sequence count 736 between the cancerous sample sequence 738 that is the same as the corresponding healthy sample sequence 734 and one or more other instances of the indel variants. For a given instance of the derived phrase 510, the count module 712 can track the cancerous sample sequence count 736 for each different instance of the corresponding cancerous sample sequence 738 in the cancer-specific data 212.

The flow can continue to the analysis module 714. The analysis module 714 may be responsible for determining whether a mutation exists in the corresponding cancerous sample sequence 738 of the cancer-specific data 212. In general, the existence of a mutation in the cancer-specific data 212 can be determined based on differences in the repeated TR patterns between the corresponding heathy sample sequence 734 and the corresponding cancerous sample sequence 738. More specifically, a difference in the number of the repeated base unit 356 can represent the existence of an indel mutation (e.g., a mutation corresponding to an insertion or a deletion of the repeated TR unit), such as for cancer-specific data 212 in comparison to the cancer-free data 210. For example, the analysis module 714 can determine that a mutation exists when the corresponding cancerous sample sequence 738 matches one of the derived segments 560 and/or the derived phrases different than that of the corresponding healthy sample sequence 734. In another example, the analysis module 714 can determine the difference between the corresponding healthy sample sequence 734 and the corresponding cancerous sample sequence 738 based on a sequence different count 740 (e.g., the total number of corresponding cancerous sample sequences 738 differing from the corresponding healthy sample sequences 734). In the case where the sequence difference count 740 indicates no differences, such as when the sequence difference count 740 is zero, the analysis module 714 can determine that no mutation exists in the corresponding cancerous sample sequence 738.

In general, the analysis module 714 can determine that an indel mutation has occurred when the sequence difference count 740 is a non-zero value. In some implementations, the analysis module 714 determines whether the indel mutation is a tumorous indel mutation based on whether the sequence difference count 740 is greater than the error percentage of the approach or apparatus used to sequence the cancer-free data 210, cancer-specific data 212, or a combination thereof.

In another implementation, the analysis module 714 can determine whether the indel mutation is a tumorous indel mutation 744 based on a tumor indication threshold 742. The tumor indication threshold 742 is an indicator of whether the number of mutations for a particular sequence in the cancer-specific data 212 indicates the existence of a tumorous indel mutation 744. The tumorous indel mutation 744 may occur when the sequence difference count 740 exceeds a tumor indication threshold 742. As an example, the tumor indication threshold 742 can be based on a percentage between the total number of sample sequence reads 730 and the sequence difference count 740. As a specific example, the tumor indication threshold 742 can require a sequence different count 740 be greater than 70 percent of the sample sequence reads 730 for the cancer-specific data 212. In another specific example, the tumor indication threshold 742 can require the sequence difference count 740 be greater than 80 percent of the sample sequence reads 730 for the cancer-specific data 212. In another specific example, the tumor indication threshold 742 can require the sequence difference count 740 be greater than 90 percent of the sample sequence reads 730 for the cancer-specific data 212.

When the corresponding cancerous sample sequence 738 includes the tumorous indel mutation 744, the computing system 100 can implement the modification module 716 to update or modify the genome TR reference catalogue 230. Said another way, the computing system 100 can implement the modification module 716 responsive to determining that the corresponding cancerous sample sequence 738 includes the tumorous indel mutation 744. For example, the modification module 716 can modify the genome TR reference catalogue 230 by identifying the instance of the catalogue entries 610 as a tumor marker 750 when the tumorous indel mutation 744 exists in the corresponding cancerous sample sequence 738.

The catalogue entries 610 that are identified as a tumor marker 750 can be modified by the modification module 716 to include tumor marker information 752. Some examples of the tumor marker information 752 can include a tumor occurrence count 754, such as the number of times that the tumorous indel mutation 744 was identified in a particular instance of the segment/phrase (e.g., TR pattern) for a given form of cancer. As a specific example, the tumor occurrence count 754 can be compiled from analysis of the DNA sample sets 206 for numerous cancer patients.

In another example, the tumor marker identification 752 can include information about the different instances of the corresponding cancerous sample sequence 738 matching to different instances of the derived segments/phrases along with the cancerous sample sequence count 736, the total number of sample sequence reads 730 of the DNA sample set 206, all or portions of the supplemental information 220, or a combination thereof. In a further example, the tumor marker information 752 can include the number of repeated base units 356 in the corresponding cancerous sample sequence 738 that were different from the corresponding healthy sample sequence 734.

The tumor marker information 752 can include information based on the supplemental information 220. For example, the tumor marker information 752 can include the supplemental information 220 (e.g., source information), such as the cancer type, the stage of cancer development, organ or tissue from which the sample was extracted, or a combination thereof. In another example, the tumor marker information 752 can include the supplemental information 220 of the patient demographic information, such as the age, the gender, the ethnicity, the geographic location of where the patient resides or has been, the duration of time that the patient stayed or resided at the geographic location, predispositions for genetic disorders or cancer development, or a combination thereof.

The computing system 100 can use one or more instances of the segments/phrases identified as the tumor marker 750 to generate the cancer correlation matrix 242 with the correlation module 718. For example, the correlation module 718 can identify cancer markers 760 based on the tumor occurrence count 754 for each of the tumor markers 750 in the genome TR reference catalogue 230. The cancer markers 760 can correspond to mutation hotspots that are specific to indel mutations in instances of the TR patterns. In one implementation, the correlation module 718 can identify the cancer markers 760 based on regression analysis. For example, the regression analysis can be performed with a receiver operating characteristic curve to the optimum sensitivity and specificity from the tumor markers 750, tumor occurrence count 754, or a combination thereof to determine the cancer markers 760.

In another implementation, the correlation module 718 can identify the cancer markers 760 based on a ratio between, or percentage of, the tumor occurrence count 754 for the tumor marker 750 and the total number of the DNA sample sets 206 of a particular form of cancer that have been analyzed for the tumor marker 750. As a specific example, the correlation module 718 can identify the cancer markers 760 as the tumor markers 750 when the ratio between the tumor occurrence count 754 and the total number of DNA sample sets 206 that are analyzed is 90 percent or more of the DNA sample sets 206 for a particular form of cancer. In this case, the cancer correlation matrix 242 can include the cancer markers 760 that were identified in this manner.

In a further implementation, the correlation module 718 generates the cancer correlation matrix 242 as the tumor markers 750 that are common among a percentage of the DNA sample sets 206 for a particular form of cancer are found. For example, the correlation module 718 can generate the cancer correlation matrix 242 as the tumor markers 750 appear in 90 percent or more of the total number of DNA sample sets 206. In other implementations, the correlation module 718 can generate the cancer correlation matrix 242 through other methods, such as regression analysis or clustering.

The correlation module 718 can generate the cancer correlation matrix 242 taking into account the supplemental information 220, such as the patient demographic information, to generate the cancer correlation matrix 242 for sub-populations. For example, the correlation module 718 can generate the cancer correlation matrix 242 based on the patient demographic information specific to gender, nationality, geographic location, occupation, age, another characteristic, or a combination of characteristics.

The computing system 100 has been described in the context of modules that perform, serve, or support certain functions as an example. The computing system 100 can partition or order the modules differently. For example, the evaluation module 710 could be implemented on the processing system 102, while the count module 712, analysis module 714, and correlation module 718 could be implemented on another computing device (also called the “external computing device” or simply “external device”) separate from the computing system. Alternatively, the processing system 102 can include the various modules described above.

The computing system 100 can implement the refinement mechanism 115 (FIG. 1A) via one or more or different modules described above. For example, the computing system 100 can include/implement the quality filter 256 in the sample evaluation module 710. Also, the computing system 100 can include/implement the consecutive overlap filter 252 and/or the duplicate filter 254 in the count module 712 (e.g., before or in preparation for the counting operations described above). Moreover, the count module 712 and/or the analysis module 714 can include the comparison correction filter 258 and/or the fraction filter 260.

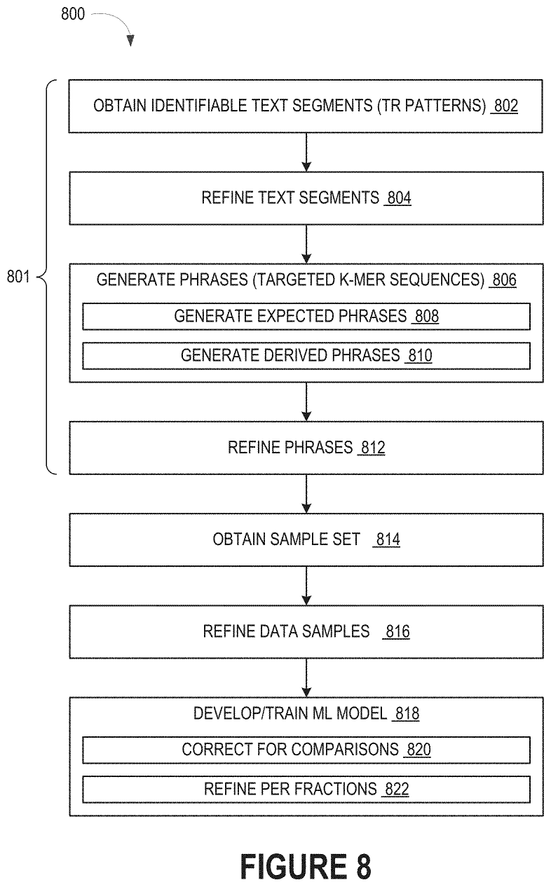

FIG. 8 shows a flow chart of a method 800 for processing and refining DNA-based text data for cancer analysis in accordance with one or more implementations of the present technology. The method 800 can be implemented using the computing system 100 (FIG. 1A) including the processing system 102 (FIG. 1A). The method 800 can be for developing the ML model 104 (FIG. 1A) including generating the various phrases and refining the processing results (via, e.g., the refinement mechanism 115 (FIG. 1)) as described above.

The method 800 includes the computing system 100 obtaining identifiable text sequences (e.g., TR-based patterns) at block 802. In some implementations, the processing system 102 can obtain the identifiable text sequences based on generating the unique segments 360 (FIG. 3) from the reference data 112 (FIG. 1A), such as by generating the character patterns representative of the identifiable TR patterns the human genome. In other implementations, the processing system 102 can access/receive the unique segments 360 generated by an external device.

The obtained unique segments 360 can serve as an initial set of segments representative of TR sequences. Each segment in the initial set can include N number of adjacently repeated base units 356. The repeated base units 356 for the initial set can have the base unit length 424 that is uniform across the segments.

At block 804, the computing system 100 can refine the identifiable text segments, such as by using/implementing the consecutive overlap filter 252 (FIG. 2). In some implementations, the processing system 102 can refine the identifiable text segments by removing the overlaps 352 (FIG. 3A), such as the TR patterns that are consecutive of and/or overlap each other, from the initial set of the unique segments 360 as described above. The processing system 102 can generate a refined set of the segments based on removing the overlaps 352 from the initial set.

At block 806, the computing system 100 can generate the phrases, such as the k-mer sequences targeted for use in subsequent data processing. For example, at block 808, the processing system 102 can generate the expected phrases 410 (FIG. 4). The processing system 102 can use the unique segments 360 (e.g., uniquely identifiable TR patterns) to generate the expected phrases 410, such as by adding different combinations of the flanking text 414 (FIG. 4) as described above. Also, at block 810, the processing system 102 can generate the derived phrases 510 (FIG. 5). The processing system 102 can use the expected phrases 410 to generate the derived phrases 510, such as by adjusting the unique segments 360 within the expected phrases to the derived segments 560 representative of indel mutations as described above.

In some implementations, the generated phrases can serve as an initial set. The generated phrases can correspond to different locations within the human genome. For example, the phrases can have the phrase length k 416 and include (1) location-specific TR-based segments (e.g., expected phrases 410) and/or (2) indel derivations of the TR-based segments adjacent to corresponding sets of flanking texts (e.g., derived phrases 510).

At block 812, the computing system 100 can refine the set of phrases, such as by using/implementing the duplicate filter 254 (FIG. 2). For example, the processing system 102 can refine the expected phrases 410 and/or derived phrases 510 by removing the duplicates or representations of DNA sequences or mutations that may correspond to more than one location. In other words, the processing system 102 can search for inadvertently generated representations of mutations that match mutations or expected/healthy sequences corresponding to a different location in the human genome as described above.

The operations described above for one or more of the blocks 802-812 can correspond to a block 801 for generating text phrases that represent different DNA sequences. The generated text phrases can represent various uniquely identifiable DNA sequences and mutations sequences for TR indel variants. The generated/refined text phrases can be used to determine correlations between the various mutations and onset cancer in the DNA sample set 206.

At block 814, the computing system 100 can obtain one or more sample sets (e.g., the DNA sample set 206 (FIG. 2)). In some implementations, the processing system 102 can receive sequenced DNA data from publicly available databases, healthcare providers, and/or submitting patients. The obtained data sample sets can include corresponding or known diagnoses, such as categorizations or tags identifying that the DNA data is from patients confirmed to be without cancer or confirmed to have specific cancers. Additionally, the obtained data can include physiological source locations of the DNA data. For samples sourced from the patients having cancer, the source locations can be the cancerous tumor or a location different from or unrelated to the malignant tumors. Accordingly, the processing system 102 can include a combination of the cancer-free data 210, the non-regional data 211, and the cancer-specific data 212, illustrated in FIG. 2. The obtained DNA sample set 112 can further include other details, such as the supplemental information 220 (FIG. 2), the sample read depth 214 (FIG. 2), the sample quality score 216 (FIG. 2), or the like.

At block 816, the computing system 100 can refine the data samples 816, such as by using/implementing the quality filter 256 (FIG. 2). For example, the processing system 102 can identify the characters corresponding to nucleotides having Phred scores less than the quality threshold. The processing system 102 can replace the identified characters with a predetermined dummy letter as described above. Additionally or alternatively, the processing system 102 can filter and/or adjust for nonuniform read counts or read depths across the DNA sample set 206. The processing system 102 can remove sample data having the sample read depth 214 below a depth requirement/threshold as described above. The processing system 102 can also adjust for the nonuniformity by calculating and applying the scale factor to the read counts as described above.

At block 818, the computing system 100 can develop and train the ML model 104 using the refined phrases and the refined data samples. For example, the processing system 102 can count and analyze the various somatic mutations, compute correlations between the mutations and cancers, and the like as described above. Using the results, the processing system 102 can select a set of features that include phrases having sufficient correlations to one or more types of cancers. The processing system 102 can design and train the ML model 104 using the selected features (e.g., correlative phrases representative of cancer-causing somatic mutations).

In developing and training the ML model 104, the processing system 102 can further refine the intermediate processing results. For example, at block 820, the processing system 102 can correct for comparison noises, such as by using/implementing the comparison correction filter 258 (FIG. 2). The processing system 102 can correct for the comparison noises using the p-value criteria as described above. Also, at block 822, the processing system 102 can refine the intermediate results per the fractional features. The processing system 102 can use the fraction filter 260 (FIG. 2) in classifying or distinguishing between somatic and non-somatic mutations.

The processing system 102 can develop/train the ML model 104 such that the model is configured to compute a cancer signal based on analyzing text-based patient DNA data according to represented somatic indel mutations in patient DNA. The processing system 102 can develop/train the ML model 104 based on computing correlations between mutations (as represented by the derived phrases) and onset/existence of one or more types of cancers as represented by the DNA sample set 206. Using the correlations, the ML model 104 can be configured to compute the cancer signal that represents (1) a likelihood that a corresponding patient has developed the one or more types of cancer, (2) a likelihood that the patient will develop the one or more types of cancer within a given duration, and/or (3) a development status at least leading up to onset of one or more types of cancer.

### Approaches to Harmonizing Genetic Analysis Across Different Extraction Kits

As discussed above, the processing system 102 can generate the cancer correlation matrix 242 of FIG. 2 or a corresponding ML model 104 that is trained to detect the existence of a particular form of cancer or indicate the possibility that a particular form of cancer will develop. Effectively, the correlation matrix 242 can represent one or more mutations in the human genome that lead to the onset of the corresponding form of cancer along with the corresponding molecular locations. Accordingly, the ML model 104 can be configured to analyze genetic information that is provided as input, to determine whether any mutations (e.g., somatic mutations) characteristic of one or more types of cancer are present. In predicting a likely future onset of cancer, the ML model 104 can be configured to identify the mutations in the genetic information via examination of the corresponding molecular locations. The ML model 104 can generate an output (also called a “signal”) based on the amount or degree of such cancer-related mutations in the genetic information. Examples of outputs include binary characterizations (e.g., yes or no, indicating whether there is evidence of cancer), “raw” numerical characterizations (e.g., the actual number of mutations found in the genetic information), “processed” numerical characterizations (e.g., a scaled version of the “raw” numerical characterization), and the like.

In applying the ML model 104, the processing system 102 can analyze genetic information to test suspicions in an unbounded manner. In other words, the ML model 104 can analyze the genetic information associated with a patient regardless of its sample source, whether derived from cancer (e.g., via tumor biopsy) or bodily fluid (e.g., via liquid biopsy). Additionally, since the ML model 104 can be trained to detect and quantify the amount or the severity of mutations in the sample taken from the patient, the processing system 102 can be unbounded as to the physiological region or pre-existing conditions of the sample source. In other words, the processing system 102 can analyze genetic information that is derived from general samples, such as blood samples, saliva samples, or the like.

As mentioned above, to identify the mutations in genetic information associated with a patient, the processing system 102 may apply an ML model 104 thereto in order to surface insights into the presence and locations of mutations. Specifically, the ML model 104 may identify whether mutations can be found at molecular locations specified in a genome tandem repeat reference catalogue 230 generated by the processing system 102. A determination regarding the presence or severity of cancer can then be determined based on the outputs, if any, produced by the ML model 104.

A potential source of error is attributable to the extraction kit that is used to extract the genetic information from DNA included in a sample taken from the patient. As mentioned above, a wide variety of extraction kits are commercially available, and these extraction kits can vary in the principles, procedures, or methodologies that are employed to extract genetic information. Simply put, different extraction kits are able to more deeply and accurate derive different sequences in the human genome.

This influences the analysis performed by the processing system 102. Consider a scenario where the processing system 102 is tasked with determining whether evidence of cancer exists in a given dataset of genetic information associated with a given patient. As discussed above, the processing system 102 may apply the ML model 104 to the given dataset, such that the ML model 104 examines the genetic information corresponding to a set of genomic positions. The set of genomic positions may correspond to the unique segment set 113, initial analysis set 114, or refined set 116. If the extraction kit does not sufficiently extract genetic information over a portion of the genome that includes one or more molecular positions in the set, outputs produced by the ML model 104 may be erroneous. For example, the processing system 102 may erroneously determine that no evidence of cancer exists based on the incomplete genetic information. As another example, the processing system 102 may predict that the given patient has one cancer when the given patient actually has another cancer whose mutations were not fully discoverable due to the incomplete genetic information.

In fact, studies on mutation calling have documented extraction kit bias effects in Whole Exome Sequencing (WES) data from The Cancer Genome Atlas (TCGA) database, hindering direct comparison between samples from different extraction kits. For example, in classification a cancer type that is exclusively samples by a first type of extraction kit in the training dataset may have very low accuracy if the testing dataset (also called the “validating dataset”) was sampled by a second type of extraction kit. To enable cross-kit, between-cancer genotype analyses with datasets like the one available from TCGA, a transformation algorithm can be developed and employed by the processing system 102 to remove extraction kit batch effects. This transformation algorithm can be tested with the ML model 104 that uses mutation markers (e.g., TR sequences) as training features. As further discussed below, this algorithm can transform data included in a given dataset that specifies read counts of various TR sequences to remove low-quality samples mitigate differences in read depth, and address extraction kit batch effects from the given dataset.

As an example, the processing system 102 may investigate the read count of different TR sequences in a training dataset (e.g., comprised of data associated with WES samples obtained from TCGA). Specifically, the processing system 102 may show that the read counts of the TR sequences do not correlate across extraction kits but correlate within capture kits. For example, the processing system 102 may calculate a Pearson correlation coefficient or a Spearman's rank correlation coefficient between the read counts of an incoming sample and read counts associated with an extraction kit. Whether a correlating extraction kit can be identified by the processing system 102 may be based on the value of these correlation coefficient (e.g., whether value equals or exceeds a threshold, such as 0.98, 0.95, or 0.92). This suggests that WES read count is largely independent from an exon's location in the genome and is more strongly correlated with the extraction kit used to generate the data. Note that in some implementations, the processing system 102 is programmed such that samples are only processed if generated by a known extraction kit. Accordingly, the processing system 102 may not be able to process a sample if generated by an extraction kit for which no information is available (e.g., in the training data).

Then, the processing system 102 can discover whether the discovery rate for TR sequences for each sample within each extraction kit is normally distributed. Outliers with very low discovery rates can be used by the processing system 102 for quality filtering. Contemporaneously, the processing system 102 can retain cancer-specific signals in the data. Before applying the transformation algorithm, the accuracy of cancer type—as predicted by the ML model 104—may be low (e.g., 0-25 percent) if the testing dataset uses a different extraction kit than the training dataset. By applying the transformation algorithm as set forth below, the processing system 102 can improve the accuracy of cancer type—as predicted by the ML model 104—to upwards of 65 percent (and possibly upwards of 80 or 90 percent) depending on the cancer type. As an example, using the approach described herein, the processing system 102 was able to achieve 93 percent sensitivity for the breast cancer gene (BRCA), whereas sensitivity was only 25 percent without the approach described herein.

Accordingly, the approach described herein can be used to address problems associated with biased classification due to the nature of the extraction kits themselves. Assume, for example, that a first type of cancer is always sequenced using a first type of extraction kit in the data used to train the ML model 104 to detect the first type of cancer. Moreover, assume that a second type of cancer is always sequenced using a second type of extraction kit in the data used to train the ML model 104 to detect the second type of cancer. After training is complete, if a sample with the second type of cancer is sequenced with the first type of extraction kit, the processing system 102 may determine—based on analysis by the ML model 104—that the corresponding patient has the first type of cancer. This erroneous conclusion may be attributed to the differences in how the first and second types of extraction kit actually extract genetic information. Rather than learn the mutations that are actually representative of the first and second types of cancer, the ML model 104 may instead learn which mutations can be surfaced using the first and second types of extraction kits due to variation (e.g., in read count, depth, etc.) in how different regions of the genome are sequenced.

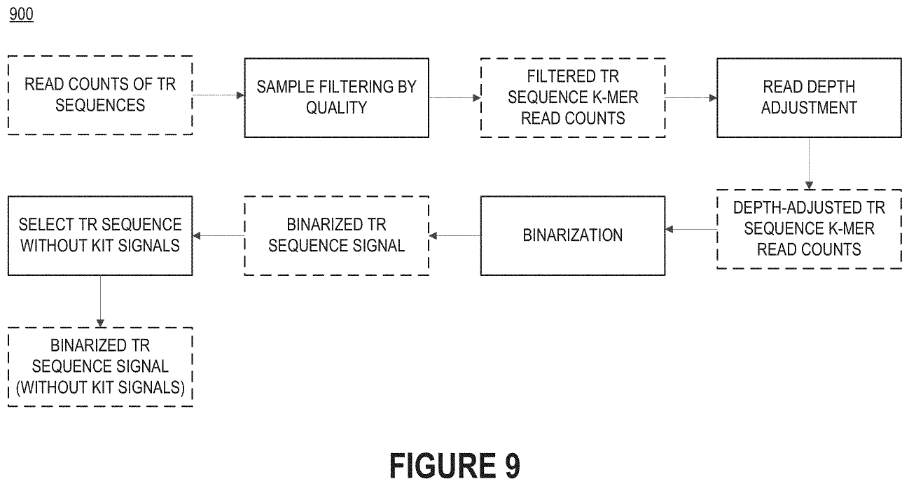

As discussed above with reference to FIG. 1C, a list of usable molecular locations can be reduced to increase the speed with which genetic information can be examined. The resulting subset of molecular locations can be processed to remove biases, unqualified samples, and the like. As part of this “pre-processing” step, the processing system 102 can eliminate kit-related biases. FIG. 9 shows a flow chart of a method 900 that, when implemented by the processing system 102, provides for removal of kit-specific signals from genetic information. As mentioned above, kit-specific signals could be removed from genetic information to which the mL model 104 is applied by the processing system 102 as part of a learning operation, or kit-specific signals could be removed from genetic information to which the ML model 104 is applied by the processing system 102 as part of an inferencing operation. Accordingly, the processing system 102 may remove kit-specific signals from genetic information to which the ML model 104 is to be applied for learning purposes or diagnosing purposes. In FIG. 9, dashed lines are used to represent data while solid lines are used to represent steps to be performed by the processing system 102. In some embodiments each step is carried out through execution of a separate algorithm, while in other embodiments each step is carried out through execution of a subroutine of a single algorithm. The steps of the method 900 are further discussed below.

### A. Sample Filtering by Quality

As shown in FIG. 9, the processing system 102 may initially receive, as input, read counts for TR sequences in samples corresponding to a given extraction kit. The read counts could be determined by the processing system 102 through analysis of genetic information corresponding to the samples as discussed above. While each sample may correspond to its own dataset of genetic information, these datasets may be collectively referred to as the “superset of data” or simply “data” that is associated with the given extraction kit and is to be used by the processing system 102 for training or inferencing purposes. Table 3 includes the read counts for k-mers of three TR sequences for two samples.

TABLE 3
Read Counts for K-mers of Exemplary TR Sequences

| TR Sequence K-mer Read Count | Sample 1 | Sample 2 |
|---|---|---|
| TR Sequence 1 +0 | 0 | 5 |
| TR Sequence 1 −1 | 0 | 10 |
| TR Sequence 2 +0 | 0 | 0 |
| TR Sequence 2 −1 | 0 | 0 |
| TR Sequence 2 +3 | 0 | 100 |
| TR Sequence 3 +0 | 0 | 27 |

In this example, all TR sequences in the first sample have zero signal. Because these TR sequences have zero signal, the first sample will not be useful for machine learning purposes—at least in terms of training the ML model to recognize TR Sequence 1 +0, TR Sequence 1 −1, TR Sequence 2 +0, TR Sequence 2 −1, TR Sequence 2 +3, and TR Sequence 3 +0. Accordingly, the goal of the processing system 102 may be to filter out “bad” samples like the first sample.

Initially, the processing system 102 can calculate the proportion of TR sequences with reads. Said another way, the processing system 102 can calculate the proportion of TR sequences for which the read count is not zero. Consider, for example, the counts shown for two samples in Table 4. Because the first sample has non-zero counts for two TR sequences (i.e., TR Sequence 1 −1 and TR Sequence 2 −1), the first sample has a proportion of 0.33. Because the second sample has non-zero counts for five TR sequences (i.e., TR Sequence 1 +0, TR Sequence 1 −1, TR Sequence 2 −1, TR Sequence 2 +3, and TR Sequence 3 +0), the second sample has a proportion of 0.83. Note that while six TR sequences are shown in Table 4, the actual number of TR sequences monitored by the processing system 102 may be in the hundreds, thousands, tens of thousands, hundreds of thousands, millions, or tens of millions in practice. Moreover, note that the proportions computed by the processing system 102 are not dependent on the number of counts, only whether the count is a non-zero value.

TABLE 4
Read Counts for K-mers of Exemplary TR Sequences

| TR Sequence K-mer Read Count | Sample 1 | Sample 2 |
|---|---|---|
| TR Sequence 1 +0 | 0 | 5 |
| TR Sequence 1 −1 | 5 | 10 |
| TR Sequence 2 +0 | 0 | 0 |
| TR Sequence 2 −1 | 10 | 2 |
| TR Sequence 2 +3 | 0 | 100 |
| TR Sequence 3 +0 | 0 | 27 |
| Proportion of TR Sequences with Reads | 0.33 | 0.83 |

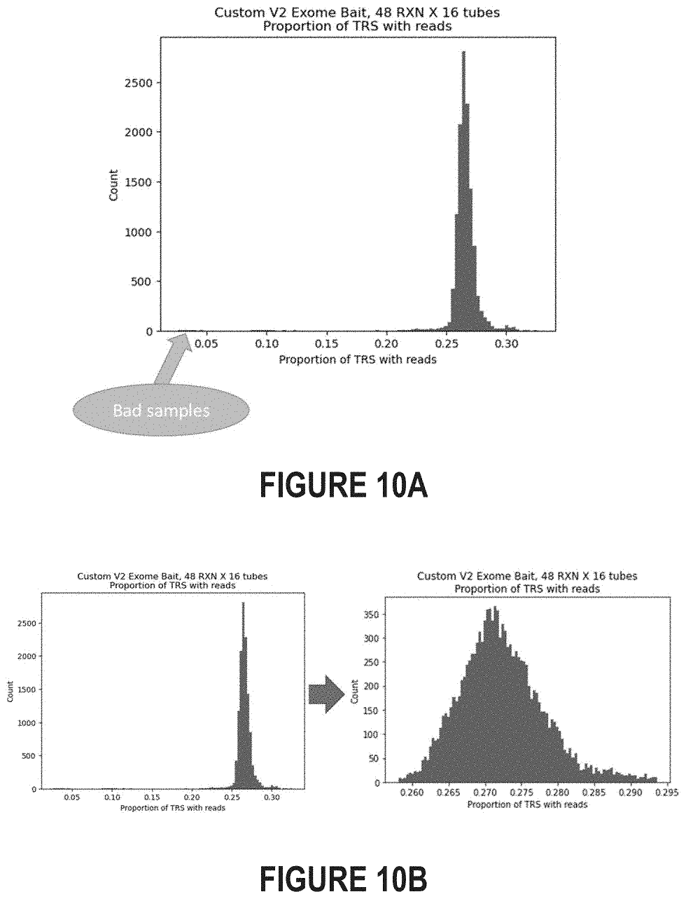

Thereafter, the processing system 102 can filter the samples using a histogram as shown in FIG. 10A. In this example, an extraction kit called “Custom V2 Exome Bait” is used to produce genetic information for the samples. Using the histogram, the processing system 102 can define a quality range in which “good” samples reside. The bounds of the quality range can be dynamically and statistically computed, for example, so as to include a predetermined percentage (e.g., 90 percent, 95 percent, 98 percent) of all samples. Alternatively, the bounds of the quality range can be dynamically and statistically computed, for example, so as to include all samples that are determined to be statistically comparable to one another. In such a scenario, the processing system 102 may apply, to the samples, a clustering algorithm that identifies outliers, if any, that should not be included in the quality range. Referring again to the example in FIG. 10A, the proportions computed for the samples are generally around 0.26-0.28, and therefore the processing system 102 may define a quality range of 0.25-0.30. To define the quality range, the processing system 102 may define a Gaussian model using the existing samples and then filter out samples based on the significance level with respect to the ML model 104.

FIG. 10B illustrates how low-quality samples outside of the quality range can be filtered from the data being processed. By doing this, the samples are more “compact” as shown in FIG. 10B, ensuring that any insights gleaned from analysis of the samples is more reliable. The quality range may be defined by a lower bound, an upper bound, or a combination of lower and upper bounds. As mentioned above, the quality range is generally defined by lower and upper bounds, that together define an upper and lower bounded range. However, the quality range could be open ended along its lower end or upper end. In scenarios where the outlier samples tend to have low proportions of TR sequences with reads, the processing system 102 could define the quality range as above a lower threshold, in which case all samples whose computed proportions fall below the lower threshold are filtered. Referring to FIG. 10B, for example, the processing system 102 could define the quality range as greater than 0.25, in which case all samples whose computed proportions fall below 0.25 are filtered. In scenarios where the outlier samples tend to have high proportions of TR sequences with reads, the processing system 102 could define the quality range as beneath an upper threshold, in which case all samples whose computed proportions exceed the upper threshold are filtered.

### B. Read Depth Adjustment

After the “bad” samples have been filtered from the data, the processing system 102 may adjust the read depth of the remaining samples. Consider Table 5 in which counts are provided for k-mers of four TR sequences across two samples.

TABLE 5
Read Counts for K-mers of Exemplary TR Sequences

| TR Sequence K-mer Read Count | Sample 1 | Sample 2 |
|---|---|---|
| TR Sequence 1 +0 | 1 | 5 |
| TR Sequence 1 −1 | 3 | 10 |
| TR Sequence 2 +0 | 0 | 0 |
| TR Sequence 2 −1 | 0 | 3 |
| TR Sequence 2 +3 | 5 | 8 |
| TR Sequence 3 +0 | 1 | 2 |
| TR Sequence 4 +0 | 0 | 1 |
| TR Sequence 4 −2 | 1 | 4 |

In this example, the first sample has a higher proportion of zero counts because of its lower read depth. As mentioned above, differences in read depth—and therefore, the number and magnitude of non-zero counts for TR sequences—occur across different extraction kits. The difference in the number of TR sequences with reads is a type of bias that should be removed, or at least mitigated as much as possible, as it can be attributed to the extraction kits. At a high level, the goal of the processing system 102 may be to ensure that all samples have the same number of TR sequences with reads, so as to mitigate kit-driven impact.

It is relatively common for a given TR sequence to be discovered in only some samples included in a large dataset (e.g., with genetic information for more than 5, 10, or 50 samples). Consider, for example, Table 6 in which counts are provided for k-mers of four TR sequences across four samples.

TABLE 6
Read Counts for K-mers of Exemplary TR Sequences

| TR Sequence | | | | | Discovery |
|---|---|---|---|---|---|
| K-mer Read Count | Sample 1 | Sample 2 | Sample 3 | Sample 4 | Rate |
| TR Sequence 1 +0 | 1 | 5 | 6 | 0 | 0.75 |
| TR Sequence 1 −1 | 3 | 10 | 11 | 2 | 1.00 |
| TR Sequence 2 +0 | 0 | 0 | 2 | 4 | 0.50 |
| TR Sequence 2 −1 | 0 | 3 | 0 | 3 | 0.50 |
| TR Sequence 2 +3 | 5 | 8 | 5 | 5 | 1.00 |
| TR Sequence 3 +0 | 1 | 2 | 0 | 0 | 0.50 |
| TR Sequence 4 +0 | 0 | 0 | 0 | 0 | 0 |
| TR Sequence 4 −2 | 0 | 0 | 0 | 6 | 0.25 |

As shown in Table 6, the processing system 102 can compute the discovery rate for each TR sequence k-mer across the entire set of samples. At a high level, the discovery rate is representation of the proportion of samples across which a given TR sequence k-mer is present. Here, for example, TR Sequence 1 +0 has a non-zero count for the first, second, and third samples, and therefore has a discovery rate of 0.75. As another example, TR Sequence 1 −1 has a non-zero count for the first, second, third, and fourth samples, and therefore has a discovery rate of 1.00. Meanwhile, TR Sequence 4 +0 has a zero count for the first, second, third, and fourth samples, and therefore has a discovery rate of 0. Like the proportion computed above, the discovery rate is not dependent on the number of counts, only whether the count is a non-zero value.

Thereafter, the processing system 102 can adjust the read depth to a target value, so as to harmonize or homogenize the samples. As an example, the read depth of all samples may be adjusted to the lowest read depth sample. Assume, for example, that there are two samples (i.e., a first sample and a second sample) as shown in Table 7. Because the proportion of TR Sequences with reads is greater for the second sample than the first sample, the second sample can be adjusted to the first sample.

TABLE 7
Read Counts for K-mers of Exemplary TR Sequences

| TR Sequence K-mer Read Count | Sample 1 | Sample 2 |
|---|---|---|
| TR Sequence 1 +0 | 0 | 5 |
| TR Sequence 1 −1 | 0 | 10 |
| TR Sequence 2 +0 | 0 | 0 |
| TR Sequence 2 −1 | 2 | 3 |
| Proportion of TR Sequences with Reads | 0.25 | 0.75 |

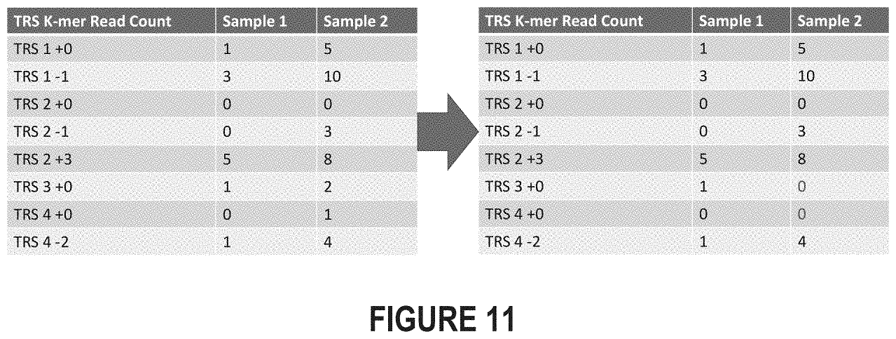

FIG. 11 illustrates how TR sequences can be selected for adjustment by the processing system 102. Generally, the TR sequences with the lowest read count are removed first. In FIG. 11, TR Sequence 3 +0 and TR Sequence 4 +0 have the lowest read counts of one and two, respectively, in the second sample. TR sequences can then be removed in order of increasing read count until the proportion of TR sequences with reads are the same. Discovery rate may be used as the tiebreaker. Accordingly, if multiple TR sequences have the same read counts, then the processing system 102 may compute the discovery rate for each of the multiple TR sequences and then remove whichever TR sequences has the lowest discovery rate. Again, if more than two TR sequences have the same read count, then TR sequences can be removed in order of increasing discovery rate until either (i) the proportion of TR sequences with reads are the same or (ii) all of the TR sequences with the same read count have been removed, in which case the process can proceed with the next lowest read count.

After this step, all samples available for the given extraction kit will have the same number of TR sequences with reads, though not necessarily the same TR sequences and k-mers. Normally, this process is performed for the samples associated with each of multiple extraction kits. Accordingly, through pre-processing, the processing system 102 can ensure that all samples associated with each of multiple extraction kits will have the same number of TR sequences with reads.

### C. Binarization

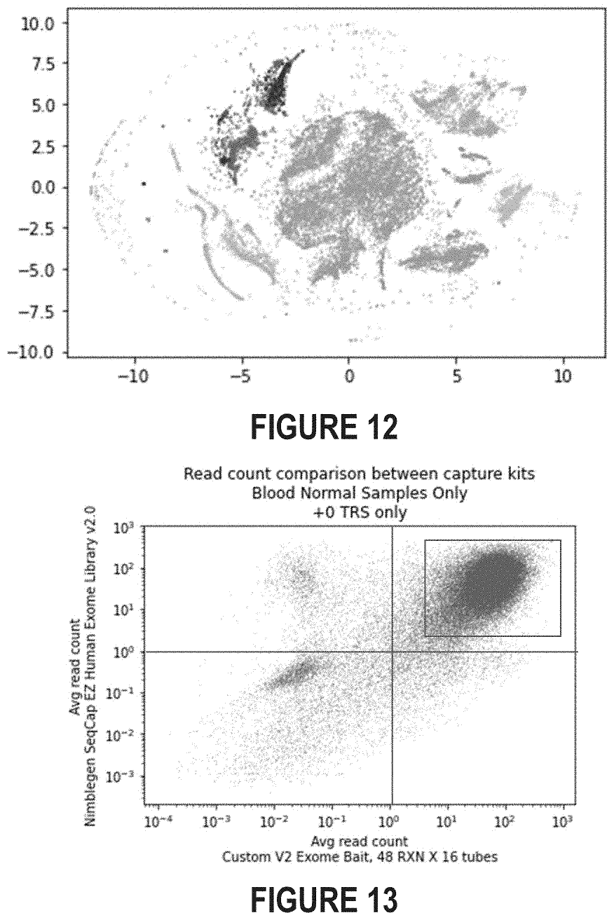

Binary data—which can be more easily handled by the processing system 102, as the necessary computational resources and time for analysis are lowered—is comprised of zeros and ones. As part of the method 900, the processing system 102 can transform the read counts of the TR sequences and k-mers into zeros and ones. Simply put, the processing system 102 can convert all non-zero counts into ones while the zero counts are left as zeros. This step—referred to as “binarization”—allows the processing system 102 to more readily compare and contrast extraction kits. FIG. 12 includes an example of a t-distributed stochastic neighbor embedding (t-SNE) plot with samples colored according to extraction kit. Said another way, the samples corresponding to each extraction kit are represented using a different color in the t-SNE plot. Such a visualization allows clusters of extraction kits to be found, thereby surfacing insights into weak and strong biases.

Rather than quantify bias, for example, as either weak or strong, the processing system 102 may instead presume that bias exists, as bias from extraction kits is a known issue. Thus, the “pipeline” employed by the processing system 102 may not involve computation of any metrics to measure the intensity of the bias. However, the processing system 102 may require that the identity of the extraction kit be known in order for the approach described herein to work, as mentioned above. In other words, if there is an incoming sample that is generated by an unknown extraction kit, the processing system 102 may not implement the approach described herein (and may simply not process the incoming sample until the extraction kit is specified).

FIG. 13 includes a plot that illustrates read counts for two extraction kits. In FIG. 13, the blue lines divide the TR sequences with an average read count greater than one from the TR sequences with an average read count less than one. Generally, TR sequences with average read counts greater than one are considered “confident.” Said another way, the processing system 102 may have greater confidence in those TR sequences with average read counts greater than one. Meanwhile, the red box identifies the distribution of read counts between the two extraction kits that looks roughly like a ball. This type of distribution indicates that the two extraction kits are not correlated with one another.

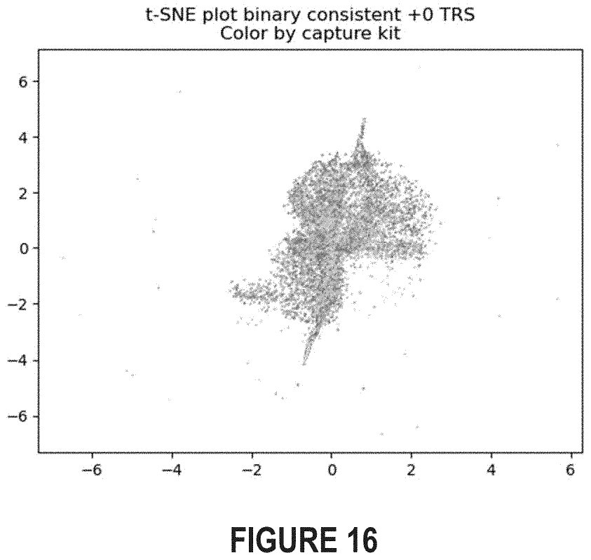

If two extraction kits correlate with each other, the read counts—when plotted against each other—would appear similar to FIG. 16 in a t-SNE plot where the samples of those two extraction kits are mixed together. Conversely, if two extraction kits do not correlate with each other, the read counts—when plotted against each other—would appear similar to FIG. 12. Note that the correlation between two extraction kits may be quantifiable by the processing system, for example, by computing a Pearson correlation coefficient or Spearman's rank correlation coefficient between the read counts of the two extraction kits.

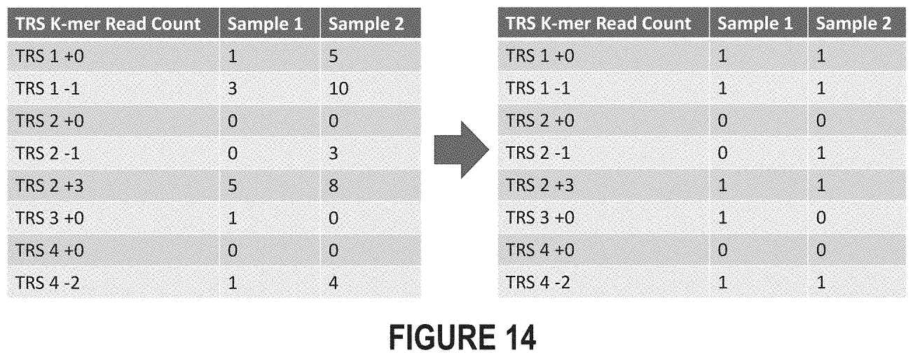

Because the two extraction kits do not correlate with one another, the read counts may be deemed to have respective extraction kit biases (or simply “kit biases”). In order to remove read count information, the processing system 102 may rely on binarization as mentioned above. FIG. 14 illustrates how all of the non-zero counts across the samples can be converted into values of one, so as to binarize the read counts.

### D. Select TR Sequences Without Kit Signals

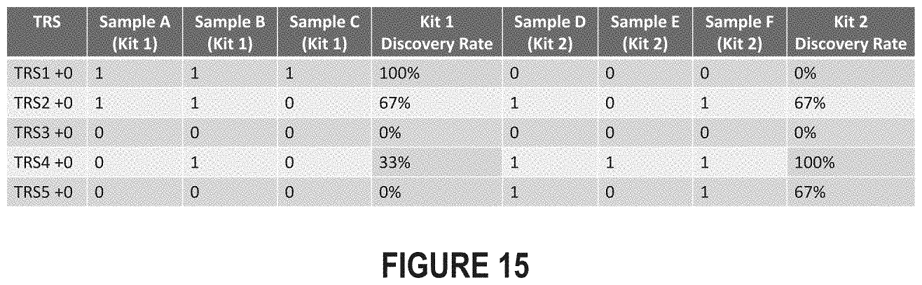

After binarization, differences between extraction kits can be detected using the discovery rate. The process for computing the discovery rate is described above. FIG. 15 shows how discovery rate can be computed on a per-kit basis across different subsets of samples. Assume, for example, that all of the samples shown in FIG. 15 correspond to the same type of cancer. By comparing the discovery rates computed for a first extraction kit (i.e., Kit 1) to the discovery rates computed for a second extraction kit (i.e., Kit 2), the processing system 102 can identify those TR sequences influenced by kit bias. Specifically, the processing system 102 can identify those TR sequences for which the discovery rates are not identical or comparable across the first and second extraction kits. In some embodiments, the discovery rates may need to be identical in order for the processing system 102 to determine that no kit bias exists. In other embodiments, the discovery rates may simply need to be comparable (e.g., within 2, 5, or 10 percent of one another) in order for the system to determine that no kit bias exists. In FIG. 15, the TR sequences determined to be affected by kit bias—due to the discovery rates of the first and second extraction kits not sufficiently matching—are highlighted.

Then, the processing system 102 can select the TR sequences for which discovery rate is consistent across the extraction kits. Said another way, the processing system 102 can identify the TR sequences for which there is no discernable kit bias. To accomplish this, the processing system 102 may execute an algorithm that programmatically implements a proportion z-test between each pair of extraction kits. A proportion z-test permits a comparison of proportions—namely, one corresponding to the first extraction kit and another corresponding to the second extraction kit—to see if those proportions are the same. The significance level (also called the “p-value”) corresponding to the z-statistic produced by the proportion z-test may also be determined by the processing system 102. FIG. 16 shows another t-SNE plot with samples colored based on extraction kit. Since clusters of samples having different colors corresponding to different extraction kits can no longer be found, it can be presumed that kit bias has been removed by the processing system 102.

The processing system 102 can store an indication of the selected text phrases in a data structure. For example, the processing system 102 may store a representation of each selected text phrase in an entry in the data structure, and the data structure may serve as a repository for the text phrases to be used for training and/or inferencing purposes. Additionally or alternatively, the processing system 102 may cause digital presentation of information related to the aforementioned steps on an interface, so as to indicate (e.g., to a user) whether bias has been detected and addressed, progress, etc. For example, the processing system 102 can cause digital presentation of an indicium that visually conveys information regarding the quality range, the filtered set of samples, the discovery rates, or the selected text phrases on an interface. Examples of visual indicia include the histograms of FIGS. 10A-B, the table of FIG. 11, the t-SNE plot of FIG. 12, the plot of FIG. 13, the tables of FIGS. 14-15, and the t-SNE plot of FIG. 16.

Note that while the approach to removing kit bias is described in the context of a pair of extraction kits—namely, the first and second extraction kits—the approach is generally applicable regardless of the number of extraction kits. Consider, for example, a scenario where the processing system 102 acquires genetic information that is derived by various extraction kits (e.g., 3, 5, or 10 extraction kits). Each of the various extraction kits may be associated with a different subset of the genetic information. In such a scenario, the processing system 102 can implement the approach, in a pairwise manner, for the various extraction kits, so as to ensure that each subset of the genetic information is “stripped” of its kit bias. One drawback of implementing the approach in a pairwise manner is that the number of remaining genetic information will continue dropping with each additional extraction kit, which may lower accuracy.

### Example of Computing System

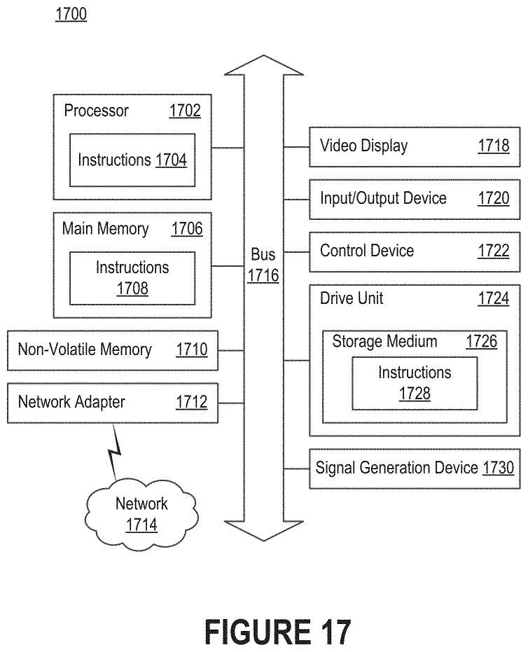

FIG. 17 is a block diagram illustrating an example of a computing system 1700 (e.g., the computing system 100 or a portion thereof, such as the processing system 102) in accordance with one or more implementations of the present technology.

The computing system 1700 may include a processor 1702, main memory 1706, non-volatile memory 1710, network adapter 1712, video display 1718, input/output device 1720, control device 1722 (e.g., a keyboard or pointing device), drive unit 1724 including a storage medium 1726, and signal generation device 1730 that are communicatively connected to a bus 1716. The bus 1716 is illustrated as an abstraction that represents one or more physical buses or point-to-point connections that are connected by appropriate bridges, adapters, or controllers. The bus 1716, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), inter-integrated circuit (I2C) bus, or an Institute of Electrical and Electronics Engineers (IEEE) standard 1394 bus (also referred to as “Firewire”).

While the main memory 1706, non-volatile memory 1710, and storage medium 1726 are shown to be a single medium, the terms “machine-readable medium” and “storage medium” should be taken to include a single medium or multiple media (e.g., a centralized/distributed database and/or associated caches and servers) that store one or more sets of instructions 1728. The terms “machine-readable medium” and “storage medium” shall also be taken to include any medium that is capable of storing, encoding, or carrying a set of instructions for execution by the computing system 1700.

In general, the routines executed to implement the embodiments of the disclosure may be implemented as part of an operating system or a specific application, component, program, object, module, or sequence of instructions (collectively referred to as “computer programs”). The computer programs typically comprise one or more instructions (e.g., instructions 1704, 1708, 1728) set at various times in various memory and storage devices in a computing device. When read and executed by the processors 1702, the instruction(s) cause the computing system 1700 to perform operations to execute elements involving the various aspects of the present disclosure.

Further examples of machine- and computer-readable media include recordable-type media, such as volatile memory devices and non-volatile memory devices 1710, removable disks, hard disk drives, and optical disks (e.g., Compact Disk Read-Only Memory (CD-ROMS) and Digital Versatile Disks (DVDs)), and transmission-type media, such as digital and analog communication links.

The network adapter 1712 enables the computing system 1700 to mediate data in a network 1714 with an entity that is external to the computing system 1700 (e.g., between the processing system 102 and the sourcing device 152) through any communication protocol supported by the computing system 1700 and the external entity. The network adapter 1712 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A method comprising:
obtaining a dataset that specifies, for each sample in a set of samples, a number of occurrences of a plurality of text phrases, each of which is representative of a different mutation that is diagnostically relevant to a given type of cancer,
wherein
(i) a first portion of the set of samples is associated with a first type of extraction kit and
(ii) a second portion of the set of samples is associated with a second type of extraction kit;
for each sample in the set of samples,
computing a proportion of the plurality of text phrases for which the number of occurrences is not zero, so as to compute a set of proportions;
determining a quality range based on an analysis of the set of proportions;
filtering, based on the quality range, the set of samples to produce a filtered set of samples;
adjusting read depth of samples in the filtered set of samples, as necessary, based on a lowest read depth in the filtered set of samples;
binarizing the filtered set of samples by converting each non-zero count to a value of one;
determining a difference between the first and second types of extraction kits through an analysis of discovery rate that is computed for each text phrase of the plurality of text phrases on a per-kit basis;
selecting at least one of the plurality of text phrases for which the discovery rate is consistent across the first and second types of extraction kit;
storing an indication of the selected text phrases in a data structure; and
causing digital presentation of an indicium that visually conveys information regarding the quality range, the filtered set of samples, the discovery rates, or the selected text phrases on an interface.

2. The method of claim 1, further comprising:
receiving an input indicative of an instruction to train a model to identify text phrases that are representative of mutations that are diagnostically relevant for the given type of cancer; and
providing the selected text phrases to the model as input, so as to produce a trained model.

3. A non-transitory medium with instructions stored thereon that, when executed by a processor of a computing device, cause the computing device to perform operations comprising:
obtaining a dataset that specifies, for each sample in a first set of samples, a number of occurrences of a plurality of text phrases, each of which is representative of a different mutation,
wherein
(i) a first portion of the first set of samples is associated with a first type of extraction kit and
(ii) a second portion of the first set of samples is associated with a second type of extraction kit;
filtering the first set of samples to produce a second set of samples that is representative of a filtered subset of the first set of samples;
binarizing the second set of samples by converting each non-zero count to a value of one;
determining a difference between the first and second types of extraction kit through an analysis of discovery rate that is computed for each text phrase of the plurality of text phrases on a per-kit basis;
selecting at least one of the plurality of text phrases for which the discovery rate is consistent across the first and second types of extraction kits; and
storing an indication of the selected text phrases in a data structure.

4. The non-transitory medium of claim 3, wherein the operations further comprise:
computing, for each sample in the first set of samples, a proportion of the plurality of text phrases for which the number of occurrences is not zero, so as to compute a set of proportions; and
determining a quality range based on an analysis of the set of proportions;
wherein said filtering is based on the quality range.

5. The non-transitory medium of claim 4, wherein the quality range is defined by a lower bound and/or an upper bound.

6. The non-transitory medium of claim 5, wherein said filtering causes samples with proportions less than the lower bound to be filtered and/or samples with proportions greater than the upper bound to be filtered.

7. The non-transitory medium of claim 3, wherein the operations further comprise:
computing, for each sample in the first set of samples, a proportion of the plurality of text phrases for which the number of occurrences is not zero, so as to compute a set of proportions; and
adjusting read depth of samples in the second set, as necessary, to correspond to a lowest read depth in the second set of samples.

8. The non-transitory medium of claim 7, wherein said adjusting comprises:
identifying, based on the proportions, a given sample having a lowest proportion of the plurality of text phrases with non-zero values,
for each other sample in the second set,
adjusting non-zero counts to zero counts for text phrases in order from lowest non-zero count to highest non-zero count, until that sample has a same number of non-zero counts as the given sample.

9. The non-transitory medium of claim 8, wherein discovery rate is used as a tiebreaking criterion in the event that text phrases have a same non-zero count.

10. The non-transitory medium of claim 8, wherein the operations further comprise:
computing, for each of the plurality of text phrases, a discovery rate by determining a proportion of the first set of samples in which that text phrase is present.

11. The non-transitory medium of claim 3, wherein the plurality of text phrases include:
(i) expected phrases corresponding to multiple molecular locations in a human genome, wherein the expected phrases corresponding to each molecular location include different combinations of flanking characters adjacent to a corresponding text segment that represents a tandem repeat (TR) sequence associated with that molecular location, and
(ii) derived phrases representative of samples mutations in the TR sequence.

12. The non-transitory medium of claim 3, wherein each text phrase is representative of a sequence of characters that, based on characters that are expected to be located in a corresponding portion of the human genome, is determined to be indicative of a mutation.

13. The non-transitory medium of claim 3, wherein said determining comprises:
for the first type of extraction kit,
identifying samples in the second set of samples that correspond to the first portion of the first set of samples,
computing, for each text phrase of the plurality of text phrases, a discovery rate by determining a proportion of the samples in which that text phrase,
for the second type of extraction kit,
identifying samples in the second set of samples that correspond to the second portion of the first set of samples,
computing, for each text phrase of the plurality of text phrases, a discovery rate by determining a proportion of the samples in which that text phrase is present,
determining text phrases, if any, for which the discovery rate computed for the first type of extraction kit does not correspond to the discovery rate computed for the second type of extraction kit, and
identifying the determined text phrases as being influenced by kit bias.

14. A computing device comprising:
a memory that includes instructions for mitigating kit-specific signals from text-based genetic information; and
wherein the instructions, when executed by a processor, cause the processor to:
obtaining a dataset that specifies, for each sample in a set of samples, a number of occurrences of text phrases that represent different deoxyribonucleic acid (DNA) sequences, wherein
(i) a first portion of the set of samples is associated with a first type of extraction kit and
(ii) a second portion of the set of samples is associated with a second type of extraction kit;
filtering the set of samples based on a quality range that is based on a proportion of the text phrases for which the number of occurrences is not zero;
binarizing the filtered set of samples by converting each non-zero count to a value of one;
determining a difference between the first and second types of extraction kits through an analysis of discovery rate that is computed for each of the text phrases on a per-kit basis; and
selecting at least one of the text phrases for which the discovery rate is consistent across the first and second types of extraction kits.

15. The computing device of claim 14,
wherein each sample in a set of samples corresponds to a patient that is known to have a given type of cancer, and
wherein the instructions further cause the processor to:
providing the selected text phrases to a model as input, so as to produce a trained model that is able to identify text phrases that are diagnostically relevant for the given type of cancer.
